(19) United States
(12) Patent Application Publication
(10) Pub. No.: US 2011/0293744 A1
(43) Pub. Date: Dec. 1, 2011

(54) BENZOXAZOLE INHIBITORS OF POLY(ADP-RIBOSE)POLYMERASE

(75) Inventors: Magdalena K. Przytulinska, Chicago, IL (US); Thomas D. Penning, Elmhurst, IL (US); Yunsong Tong, Libertyville, IL (US)

(73) Assignee: ABBOTT LABORATORIES, Abbott Park, IL (US)

(21) Appl. No.: 12/689,297

(22) Filed: Jan. 19, 2010

Related U.S. Application Data
(60) Provisional application No. 61/145,641, filed on Jan. 19, 2009.

(51) Int. Cl. A61K 31/423; C07D 413/10; A61K 31/4439; A61K 31/454; A61P 35/00; A61K 31/475; A61K 33/24; A61K 31/513; A61K 31/7068; A61K 31/519; A61K 38/14; C07D 263/57; A61K 31/675

(52) U.S. Cl. 424/649; 548/224; 546/271.7; 546/198; 514/375; 514/338; 514/321; 514/110; 514/274; 514/49; 514/249; 514/19.3; 514/283

(57) ABSTRACT

Inhibitors of poly(ADP-ribose)polymerase having a structure of Formula (I), ways to make them and methods of treating patients using them are disclosed.

### CROSS-REFERENCE TO RELATED APPLICATIONS

This application claims priority to U.S. provisional application Ser. No. 61/145,641, filed Jan. 19, 2009.

### FIELD OF THE INVENTION

This invention relates to inhibitors of poly(ADP-ribose)polymerase, ways to make them and methods of treating patients using them.

### BACKGROUND OF THE INVENTION

Poly(ADP-ribose)polymerase (PARP) is essential for facilitating DNA repair, controlling RNA transcription, mediating cell death and regulating immune response. This activity makes PARP inhibitors targets for a number of disorders. PARP inhibitors have shown utility for treating diseases such as ischemia reperfusion injury, inflammatory disease, retroviral infections, ischemia reperfusion injury, myocardial infarction, stroke and other neural trauma, organ transplantation, reperfusion of the eye, kidney, gut and skeletal muscle, arthritis, gout, inflammatory bowel disease, CNS inflammation such as MS and allergic encephalitis, sepsis, septic shock, hemmorhagic shock, pulmonary fibrosis, and uveitis, diabetes and Parkinsons disease, liver toxicity following acetominophen overdose, cardiac and kidney toxicities from doxorubicin and platinum-based antineoplastic agents and skin damage secondary to sulfur mustards. PARP inhibitors have also been shown to potentiate radiation and chemotherapy by increasing cell death of cancer cells, limiting tumor growth, decreasing metastasis, and prolonging the survival of tumor-bearing animals. WO9524379 describes benzoxazole compounds as inhibitors of PARP.

WO2002051821 describes compounds having the general structure:

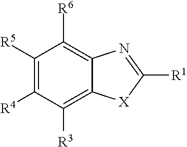

as ER-β-selective ligands.
US20060046968 describes compounds having the general structure:

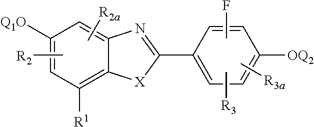

as prodrug estrogenic agents.
Demyttenaere-Kovatcheva et al., describes diphenolic axoles as ligands for estrogen receptors alpha and beta by T (J. Med. Chem. (2005) 48: 7328-7636).
Malamas et al., describes aryl diphenolic azoles asestrogen receptor-beta ligands. (J. Med. Chem. 2004 (47): 5021-5040).
There is therefore a need in the therapeutic arts for PARP inhibitors. Such compounds can be used to treat subjects suffering from cancer, and can further expand the range of treatment options available for such subjects.

### SUMMARY OF THE INVENTION

The present invention has numerous embodiments. One embodiment of this invention, therefore, pertains to compounds that inhibit the activity of poly(ADP-ribose) polymerase and have Formula I

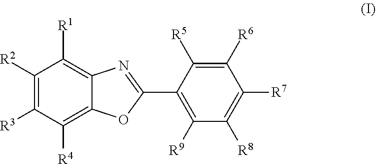

wherein

one of R¹ or R⁴ is —C(O)NH₂ and the other is selected from the group consisting of hydrogen, alkyl, halogen, cyano, haloalkyl, hydroxyalkyl, —OR¹⁶, and —NR¹⁷R¹⁸;

when R¹ is —C(O)NH₂; R⁷ is aryl, heterocyclyl, cycloalkyl, cycloalkenyl, alkylamino, alkenyl, alkynyl, halogen, cyano, —C(O)R¹⁰, —C(O)OR¹⁰, —OC(O)R¹⁰, —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —NO₂, —OCF₃, and —OCF₂CF₃;

when R⁴ is —C(O)NH₂; R⁷ is aryl, heterocyclyl, cycloalkyl, cycloalkenyl, alkylamino, alkenyl, alkynyl, halogen, cyano, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃;

R², R³ are independently selected from the group consisting of hydrogen, alkyl, alkenyl, alkynyl, halogen, cyano, haloalkoxy, haloalkyl, hydroxyalkyl, —OR¹⁶, —C(O)OR¹⁶, —NR¹⁷R¹⁸, and —C(O)NR¹⁷R¹⁸;

R⁵, R⁶, R⁸, and R⁹ are independently selected from the group consisting of hydrogen, alkyl, alkenyl, alkynyl, halogen, cyano, haloalkoxy, haloalkyl, hydroxyalkyl, —OR¹⁹, —C(O)OR¹⁹, —NR²⁰R²¹, and —C(O)NR²⁰R²¹;

R¹⁰, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo;

R¹¹ and R¹², at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;

R¹³, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo;

R¹⁴ and R¹⁵, at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;

R¹⁶, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and

R¹⁷ and R¹⁸, at each occurrence, are independently selected from the group consisting of hydrogen, alkyl, alkenyl, alkynyl, aryl, heterocyclyl, cycloalkenyl, or cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo.

R¹⁹, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and

R²⁰ and R²¹, at each occurrence, are independently selected from the group consisting of hydrogen, alkyl, alkenyl, alkynyl, aryl, heterocyclyl, cycloalkenyl, or cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo

and pharmaceutically acceptable salts thereof.

Another embodiment pertains to a compound selected from the group consisting of
2-(4-bromophenyl)-1,3-benzoxazole-4-carboxamide;
2-(1,1′-biphenyl-4-yl)-1,3-benzoxazole-4-carboxamide;
2-(4-pyridin-3-ylphenyl)-1,3-benzoxazole-4-carboxamide;
2-(4-bromophenyl)-1,3-benzoxazole-7-carboxamide;
2-(1,1′-biphenyl-4-yl)-1,3-benzoxazole-7-carboxamide;
2-[4-(1,3-benzodioxol-5-yl)phenyl]-1,3-benzoxazole-7-carboxamide;
2-[4-(1H-pyrazol-3-yl)phenyl]-1,3-benzoxazole-7-carboxamide;
2-(3′-amino-1,1′-biphenyl-4-yl)-1,3-benzoxazole-7-carboxamide;
2-(4-pyridin-3-ylphenyl)-1,3-benzoxazole-7-carboxamide;
2-(4-pyridin-4-ylphenyl)-1,3-benzoxazole-7-carboxamide;
2-(4-pyridin-2-ylphenyl)-1,3-benzoxazole-7-carboxamide;
2-phenyl-1,3-benzoxazole-4-carboxamide; and
2-(4-piperidin-3-ylphenyl)-1,3-benzoxazole-7-carboxamide;

and therapeutically acceptable salts thereof.

Another embodiment comprises the compounds:
2-(4-bromophenyl)-1,3-benzoxazole-7-carboxamide;
2-(1,1′-biphenyl-4-yl)-1,3-benzoxazole-7-carboxamide;
2-[4-(1,3-benzodioxol-5-yl)phenyl]-1,3-benzoxazole-7-carboxamide;
2-[4-(1H-pyrazol-3-yl)phenyl]-1,3-benzoxazole-7-carboxamide;
2-(3′-amino-1,1′-biphenyl-4-yl)-1,3-benzoxazole-7-carboxamide;
2-(4-pyridin-3-ylphenyl)-1,3-benzoxazole-7-carboxamide;
2-(4-pyridin-4-ylphenyl)-1,3-benzoxazole-7-carboxamide;
2-(4-pyridin-2-ylphenyl)-1,3-benzoxazole-7-carboxamide; and
2-(4-piperidin-3-ylphenyl)-1,3-benzoxazole-7-carboxamide;

and therapeutically acceptable salts thereof.

Another embodiment comprises pharmaceutical compositions comprising a compound having formula I or a pharmaceutically acceptable salt thereof and a pharmaceutically acceptable carrier. This invention also is directed to a use of one or more compounds and/or salts of the invention to prepare a medicament for the treatment of cancer. Still another embodiment comprises the use of use of one or more compounds and/or salts of Formula I for the preparation of a medicament for the treatment of leukemia, colon cancer, glioblastomas, lymphomas, melanomas, carcinomas of the breast, carcinomas of the prostate or cervical carcinomas.

Still another embodiment comprises methods of inhibiting PARP in a mammal comprising administering thereto a therapeutically acceptable amount of a compound having formula I. Still another embodiment comprises methods of treating cancer in a mammal comprising administering thereto a therapeutically acceptable amount of a compound having formula I. Still another embodiment comprises methods for decreasing tumor volume in a mammal comprising administering thereto a therapeutically acceptable amount of a compound having formula I.

Still another embodiment comprises a method of treating leukemia, colon cancer, glioblastomas, lymphomas, melanomas, carcinomas of the breast, carcinomas of the prostate or cervical carcinomas in a mammal comprising administering thereto a therapeutically acceptable amount of a compound having formula I. Still another embodiment comprises methods for potentiation of cytotoxic cancer therapy in a mammal comprising administering thereto a therapeutically acceptable amount of a compound having formula I. Still another embodiment comprises methods for potentiation of radiation therapy in a mammal comprising administering thereto a therapeutically acceptable amount of a compound having formula I.

### DETAILED DESCRIPTION OF THE INVENTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### A. Abbreviations and Definitions

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Furthermore, the use of the term “including”, as well as other forms, such as “includes” and “included”, is not limiting. With reference to the use of the words “comprise” or “comprises” or “comprising” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below. For a variable that occurs more than one time in any substituent or in the compound of the invention or any other formulae herein, its definition on each occurrence is independent of its definition at every other occurrence. Combinations of substituents are permissible only if such combinations result in stable compounds. As used in the specification and the appended claims, unless specified to the contrary, the following terms have the meaning indicated:

It is meant to be understood that proper valences are maintained for all combinations herein, that monovalent moieties having more than one atom are attached through their left ends, and that divalent moieties are drawn from left to right.

The term “alkyl” (alone or in combination with another term(s)) means a straight- or branched-chain saturated hydrocarbyl substituent typically containing from 1 to about 20 carbon atoms; or in another embodiment, from 1 to about 10 carbon atoms; from 1 to about 8 carbon atoms; in another embodiment, from 1 to about 6 carbon atoms; in another embodiment, from 1 to about 4 carbon atoms; and in another embodiment, from 1 to about 2 carbon atoms. Examples of such substituents include methyl, ethyl, n-propyl, isopropyl, n-butyl, isobutyl, sec-butyl, tert-butyl, pentyl, iso-amyl, and hexyl and the like.

The term “alkenyl” (alone or in combination with another term(s)) means a straight- or branched-chain hydrocarbyl substituent containing one or more double bonds and typically from 2 to about 20 carbon atoms; or in another embodiment, from 2 to about 8 carbon atoms; in another embodiment, from 2 to about 6 carbon atoms; and in another embodiment, from 2 to about 4 carbon atoms. Examples of such substituents include ethenyl (vinyl), 2-propenyl, 3-propenyl, 1,4-pentadienyl, 1,4-butadienyl, 1-butenyl, 2-butenyl, and 3-butenyl and the like. The term “alkynyl” (alone or in combination with another term(s)) means a straight- or branched-chain hydrocarbyl substituent containing one or more triple bonds and typically from 2 to about 20 carbon atoms; or in another embodiment, from 2 to about 8 carbon atoms; in another embodiment, from 2 to about 6 carbon atoms; and in another embodiment, from 2 to about 4 carbon atoms. Examples of such substituents include ethynyl, 2-propynyl, 3-propynyl, 2-butynyl, and 3-butynyl and the like.

The term “carbocyclyl” (alone or in combination with another term(s)) means a saturated cyclic (i.e., “cycloalkyl”), partially saturated cyclic (i.e., “cycloalkenyl”), or completely unsaturated (i.e., “aryl”) hydrocarbyl substituent containing from 3 to 14 carbon ring atoms (“ring atoms” are the atoms bound together to form the ring or rings of a cyclic substituent).

A carbocyclyl may be a single-ring (monocyclic) or polycyclic ring structure.

A carbocyclyl may be a single ring structure, which typically contains from 3 to 7 ring atoms, more typically from 3 to 6 ring atoms, and even more typically 5 to 6 ring atoms. Examples of such single-ring carbocyclyls include cyclopropyl (cyclopropanyl), cyclobutyl (cyclobutanyl), cyclopentyl (cyclopentanyl), cyclopentenyl, cyclopentadienyl, cyclohexyl (cyclohexanyl), cyclohexenyl, cyclohexadienyl, and phenyl. A carbocyclyl may alternatively be polycyclic (i.e., may contain more than one ring). Examples of polycyclic carbocyclyls include bridged, fused, and spirocyclic carbocyclyls. In a spirocyclic carbocyclyl, one atom is common to two different rings. An example of a spirocyclic carbocyclyl is spiropentanyl. In a bridged carbocyclyl, the rings share at least two common non-adjacent atoms. Examples of bridged carbocyclyls include bicyclo[2.2.1]heptanyl, bicyclo[2.2.1]hept-2-enyl, and adamantanyl. In a fused-ring carbocyclyl system, two or more rings may be fused together, such that two rings share one common bond. Examples of two- or three-fused ring carbocyclyls include naphthalenyl, tetrahydronaphthalenyl (tetralinyl), indenyl, indanyl (dihydroindenyl), anthracenyl, phenanthrenyl, and decalinyl.

The term “cycloalkyl” (alone or in combination with another term(s)) means a saturated cyclic hydrocarbyl substituent containing from 3 to 14 carbon ring atoms. A cycloalkyl may be a single carbon ring, which typically contains from 3 to 7 carbon ring atoms and more typically from 3 to 6 ring atoms. Examples of single-ring cycloalkyls include cyclopropyl, cyclobutyl, cyclopentyl, and cyclohexyl. A cycloalkyl may alternatively be polycyclic or contain more than one ring. Examples of polycyclic cycloalkyls include bridged, fused, and spirocyclic carbocyclyls.

The term “aryl” (alone or in combination with another term(s)) means an aromatic carbocyclyl containing from 6 to 14 carbon ring atoms. Examples of aryls include phenyl, naphthalenyl, and indenyl.

In some instances, the number of carbon atoms in a hydrocarbyl substituent (e.g., alkyl, alkenyl, alkynyl, or cycloalkyl) is indicated by the prefix “Cₓ-C_{y}-”, wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl means a saturated hydrocarbyl ring containing from 3 to 6 carbon ring atoms.

The term “hydrogen” (alone or in combination with another term(s)) means a hydrogen radical, and may be depicted as —H.

The term “hydroxy” (alone or in combination with another term(s)) means —OH.

The term “carboxy” (alone or in combination with another term(s)) means —C(O)—OH.

The term “amino” (alone or in combination with another term(s)) means —NH₂.

The term “halogen” or “halo” (alone or in combination with another term(s)) means a fluorine radical (which may be depicted as —F), chlorine radical (which may be depicted as —Cl), bromine radical (which may be depicted as —Br), or iodine radical (which may be depicted as —I).

A substituent is “substitutable” if it comprises at least one carbon or nitrogen atom that is bonded to one or more hydrogen atoms. Thus, for example, hydrogen, halogen, and cyano do not fall within this definition. In addition, a sulfur atom in a heterocyclyl containing such atom is substitutable with one or two oxo substituents.

If a substituent is described as being “substituted”, a non-hydrogen radical is in the place of hydrogen radical on a carbon or nitrogen of the substituent. Thus, for example, a substituted alkyl substituent is an alkyl substituent in which at least one non-hydrogen radical is in the place of a hydrogen radical on the alkyl substituent. To illustrate, monofluoroalkyl is alkyl substituted with a fluoro radical, and difluoroalkyl is alkyl substituted with two fluoro radicals. It should be recognized that if there are more than one substitution on a substituent, each non-hydrogen radical may be identical or different (unless otherwise stated).

If a substituent is described as being “optionally substituted”, the substituent may be either (1) not substituted or (2) substituted. If a substituent is described as being optionally substituted with up to a particular number of non-hydrogen radicals, that substituent may be either (1) not substituted; or (2) substituted by up to that particular number of non-hydrogen radicals or by up to the maximum number of substitutable positions on the substituent, whichever is less. Thus, for example, if a substituent is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

This patent application uses the terms “substituent” and “radical” interchangeably.

The prefix “halo” indicates that the substituent to which the prefix is attached is substituted with one or more independently selected halogen radicals. For example, haloalkyl means an alkyl substituent in which at least one hydrogen radical is replaced with a halogen radical. Examples of haloalkyls include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated).

The prefix “perhalo” indicates that every hydrogen radical on the substituent to which the prefix is attached is replaced with independently selected halogen radicals, i.e., each hydrogen radical on the substituent is replaced with a halogen radical. If all the halogen radicals are identical, the prefix typically will identify the halogen radical. Thus, for example, the term “perfluoro” means that every hydrogen radical on the substituent to which the prefix is attached is substituted with a fluorine radical. To illustrate, the term “perfluoroalkyl” means an alkyl substituent wherein a fluorine radical is in the place of each hydrogen radical.

The term “carbonyl” (alone or in combination with another term(s)) means —C(O)—.

The term “aminocarbonyl” (alone or in combination with another term(s)) means —C(O)—NH₂.

The term “oxy” (alone or in combination with another term(s)) means an ether substituent, and may be depicted as —O—.

The term “alkyloxy” (alone or in combination with another term(s)) means an alkylether substituent, i.e., —O-alkyl. Examples of such a substituent include methoxy (—O—CH₃), ethoxy, n-propoxy, isopropoxy, n-butoxy, iso-butoxy, sec-butoxy, and tert-butoxy.

The term “alkylcarbonyl” (alone or in combination with another term(s)) means —C(O)-alkyl.

The term “aminoalkylcarbonyl” (alone or in combination with another term(s)) means —C(O)-alkyl-NH₂.

The term “alkyloxycarbonyl” (alone or in combination with another term(s)) means —C(O)—O-alkyl.

The term “carbocyclylcarbonyl” (alone or in combination with another term(s)) means —C(O)-carbocyclyl.

Similarly, the term “heterocyclylcarbonyl” (alone or in combination with another term(s)) means —C(O)-heterocyclyl.

The term “carbocyclylalkylcarbonyl” (alone or in combination with another term(s)) means —C(O)-alkyl-carbocyclyl.

Similarly, the term “heterocyclylalkylcarbonyl” (alone or in combination with another term(s)) means —C(O)-alkyl-heterocyclyl.

The term “carbocyclyloxycarbonyl” (alone or in combination with another term(s)) means —C(O)—O-carbocyclyl.

The term “carbocyclylalkyloxycarbonyl” (alone or in combination with another term(s)) means —C(O)—O-alkyl-carbocyclyl.

The term “thio” or “thia” (alone or in combination with another term(s)) means a thiaether substituent, i.e., an ether substituent wherein a divalent sulfur atom is in the place of the ether oxygen atom. Such a substituent may be depicted as —S—. This, for example, “alkyl-thio-alkyl” means alkyl-S-alkyl (alkyl-sulfanyl-alkyl).

The term “thiol” or “sulfhydryl” (alone or in combination with another term(s)) means a sulfhydryl substituent, and may be depicted as —SH.

The term “(thiocarbonyl)” (alone or in combination with another term(s)) means a carbonyl wherein the oxygen atom has been replaced with a sulfur. Such a substituent may be depicted as —C(S)—.

The term “sulfonyl” (alone or in combination with another term(s)) means —S(O)₂—.

The term “aminosulfonyl” (alone or in combination with another term(s)) means —S(O)₂—NH₂.

The term “sulfinyl” or “sulfoxido” (alone or in combination with another term(s)) means —S(O)—.

The term “heterocyclyl” (alone or in combination with another term(s)) means a saturated (i.e., “heterocycloalkyl”), partially saturated (i.e., “heterocycloalkenyl”), or completely unsaturated (i.e., “heteroaryl”) ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur. A heterocyclyl may be a single-ring (monocyclic) or polycyclic ring structure.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, dihydrofuranyl, tetrahydrofuranyl, thiophenyl (thiofuranyl), dihydrothiophenyl, tetrahydrothiophenyl, pyrrolyl, pyrrolinyl, pyrrolidinyl, imidazolyl, imidazolinyl, imidazolidinyl, pyrazolyl, pyrazolinyl, pyrazolidinyl, triazolyl, tetrazolyl, oxazolyl, oxazolidinyl, isoxazolidinyl, isoxazolyl, thiazolyl, isothiazolyl, thiazolinyl, isothiazolinyl, thiazolidinyl, isothiazolidinyl, thiodiazolyl, oxadiazolyl (including 1,2,3-oxadiazolyl, 1,2,4-oxadiazolyl, 1,2,5-oxadiazolyl (furazanyl), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl, dihydropyranyl, thiopyranyl, tetrahydrothiopyranyl, pyridinyl (azinyl), piperidinyl, diazinyl (including pyridazinyl (1,2-diazinyl), pyrimidinyl (1,3-diazinyl), or pyrazinyl (1,4-diazinyl)), piperazinyl, triazinyl (including 1,3,5-triazinyl, 1,2,4-triazinyl, and 1,2,3-triazinyl)), oxazinyl (including 1,2-oxazinyl, 1,3-oxazinyl, or 1,4-oxazinyl)), oxathiazinyl (including 1,2,3-oxathiazinyl, 1,2,4-oxathiazinyl, 1,2,5-oxathiazinyl, or 1,2,6-oxathiazinyl)), oxadiazinyl (including 1,2,3-oxadiazinyl, 1,2,4-oxadiazinyl, 1,4,2-oxadiazinyl, or 1,3,5-oxadiazinyl)), morpholinyl, azepinyl, oxepinyl, thiepinyl, and diazepinyl.

A heterocyclyl may alternatively be polycyclic (i.e., may contain more than one ring). Examples of polycyclic heterocyclyls include bridged, fused, and spirocyclic heterocyclyls. In a spirocyclic heterocyclyl, one atom is common to two different rings. In a bridged heterocyclyl, the rings share at least two common non-adjacent atoms. In a fused-ring heterocyclyl, two or more rings may be fused together, such that two rings share one common bond. Examples of fused ring heterocyclyls containing two or three rings include indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl. Other examples of fused-ring heterocyclyls include benzo-fused heterocyclyls, such as indolyl, isoindolyl (isobenzazolyl, pseudoisoindolyl), indoleninyl (pseudoindolyl), isoindazolyl (benzpyrazolyl), benzazinyl (including quinolinyl (1-benzazinyl) or isoquinolinyl (2-benzazinyl)), phthalazinyl, quinoxalinyl, quinazolinyl, benzodiazinyl (including cinnolinyl (1,2-benzodiazinyl) or quinazolinyl (1,3-benzodiazinyl)), benzopyranyl (including chromanyl or isochromanyl), benzoxazinyl (including 1,3,2-benzoxazinyl, 1,4,2-benzoxazinyl, 2,3,1-benzoxazinyl, or 3,1,4-benzoxazinyl), and benzisoxazinyl (including 1,2-benzisoxazinyl or 1,4-benzisoxazinyl).

The term “heteroaryl” (alone or in combination with another term(s)) means an aromatic heterocyclyl containing from 5 to 14 ring atoms. A heteroaryl may be a single ring or 2 or 3 fused rings. Examples of heteroaryl substituents include 6-membered ring substituents such as pyridyl, pyrazyl, pyrimidinyl, pyridazinyl, and 1,3,5-, 1,2,4- or 1,2,3-triazinyl; 5-membered ring substituents such as imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, benzisoxazolyl, benzoxazolyl, purinyl, and anthranilyl; and 6/6-membered fused rings such as benzopyranyl, quinolinyl, isoquinolinyl, cinnolinyl, quinazolinyl, and benzoxazinyl.

A prefix attached to a multi-component substituent only applies to the first component. To illustrate, the term “alkylcycloalkyl” contains two components: alkyl and cycloalkyl. Thus, the C₁-C₆-prefix on C₁-C₆-alkylcycloalkyl means that the alkyl component of the alkylcycloalkyl contains from 1 to 6 carbon atoms; the C₁-C₆-prefix does not describe the cycloalkyl component. To illustrate further, the prefix “halo” on haloalkyloxyalkyl indicates that only the alkyloxy component of the alkyloxyalkyl substituent is substituted with one or more halogen radicals. If halogen substitution may alternatively or additionally occur on the alkyl component, the substituent would instead be described as “halogen-substituted alkyloxyalkyl” rather than “haloalkyloxyalkyl.” And finally, if the halogen substitution may only occur on the alkyl component, the substituent would instead be described as “alkyloxyhaloalkyl.”

The term “pharmaceutically acceptable carrier” refers to a carrier that is compatible with the other ingredients in the composition and is not deleterious to the subject. Such carriers may be pharmaceutically acceptable material, compositions, or vehicles such as liquid or solid filler, diluent, excipient, solvent or encapsulating material, involved in carrying or transporting a chemical agent.

The term “therapeutically acceptable amount” refers to that amount of drug or pharmaceutical agent that will elicit the biological or medical response of a tissue, system or animal that is being sought be a researcher or clinician.

The term “treatment” (and corresponding terms “treat” and “treating”) includes palliative, restorative, and preventative treatment of a subject. The term “palliative treatment” refers to treatment that eases or reduces the effect or intensity of a condition in a subject without curing the condition. The term “preventative treatment” (and the corresponding term “prophylactic treatment”) refers to treatment that prevents the occurrence of a condition in a subject. The term “restorative treatment” refers to treatment that halts the progression of, reduces the pathologic manifestations of, or entirely eliminates a condition in a subject.

### B. Compounds

In one embodiment, the present invention is directed, in part, to a class of compounds having a structure of Formula I

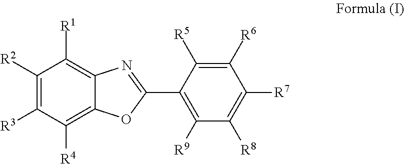

wherein

one of R¹ or R⁴ is —C(O)NH₂ and the other is selected from the group consisting of hydrogen, alkyl, halogen, cyano, haloalkyl, hydroxyalkyl, —OR¹⁶, and —NR¹⁷R¹⁸;

when R¹ is —C(O)NH₂; R⁷ is aryl, heterocyclyl, cycloalkyl, cycloalkenyl, alkylamino, alkenyl, alkynyl, halogen, cyano, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —NO₂, —OCF₃, and —OCF₂CF₃;

when R⁴ is —C(O)NH₂; R⁷ is aryl, heterocyclyl, cycloalkyl, cycloalkenyl, alkylamino, alkenyl, alkynyl, halogen, cyano, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃;

R², R³ are independently selected from the group consisting of hydrogen, alkyl, alkenyl, alkynyl, halogen, cyano, haloalkoxy, haloalkyl, hydroxyalkyl, —OR¹⁶, —C(O)OR¹⁶, —NR¹⁷R¹⁸, and —C(O)NR¹⁷R¹⁸;

R⁵, R⁶, R⁸, and R⁹ are independently selected from the group consisting of hydrogen, alkyl, alkenyl, alkynyl, halogen, cyano, haloalkoxy, haloalkyl, hydroxyalkyl, —OR¹⁹, —C(O)OR¹⁹, —NR²⁰R²¹, and —C(O)NR²⁰R²¹;

R¹⁰, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo;

R¹¹ and R¹², at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;

R¹³, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo;

R¹⁴ and R¹⁵, at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;

R¹⁶, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and

R¹⁷ and R¹⁸, at each occurrence, are independently selected from the group consisting of hydrogen, alkyl, alkenyl, alkynyl, aryl, heterocyclyl, cycloalkenyl, or cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo

R¹⁹, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and

R²⁰ and R²¹, at each occurrence, are independently selected from the group consisting of hydrogen, alkyl, alkenyl, alkynyl, aryl, heterocyclyl, cycloalkenyl, or cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo.

In one embodiment of Formula I, R⁵, R⁶, R⁸, and R⁹ are hydrogen, wherein R² and R³ are as described in Formula I. In another embodiment of Formula I, R² and R³ are hydrogen, as described in Formula II:

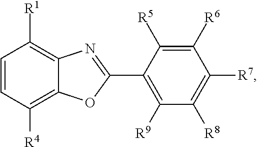

wherein one of R¹ or R⁴ is —C(O)NH₂ and the other is hydrogen, and R¹, R⁴, R⁵, R⁶, R⁷, R⁸ and

R⁹ are as described in Formula I.

### Formula II

In one embodiment, the present invention is directed, in part, to a class of compounds having a structure of Formula II,

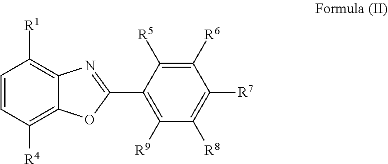

wherein

one of R¹ or R⁴ is —C(O)NH₂ and the other is hydrogen;

when R¹ is —C(O)NH₂; R⁷ is aryl, heterocyclyl, cycloalkyl, cycloalkenyl, alkylamino, alkenyl, alkynyl, halogen, cyano, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —NO₂, —OCF₃, and —OCF₂CF₃;

when R⁴ is —C(O)NH₂; R⁷ is aryl, heterocyclyl, cycloalkyl, cycloalkenyl, alkylamino, alkenyl, alkynyl, halogen, cyano, —C(O)R¹³, —C(O)OR¹³, —OC(O)R¹³—NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃;

R⁵, R⁶, R⁸, and R⁹ are independently selected from the group consisting of hydrogen, alkyl, alkenyl, alkynyl, halogen, cyano, haloalkoxy, haloalkyl, hydroxyalkyl, —OR¹⁹, —C(O)OR¹⁹, —NR²⁰R²¹, and —C(O)NR²⁰R²¹;

R¹⁰, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo;

R¹¹ and R¹², at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;

R¹³, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo;

R¹⁴ and R¹⁵, at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;

R¹⁹, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and

R²⁰ and R²¹, at each occurrence, are independently selected from the group consisting of hydrogen, alkyl, alkenyl, alkynyl, aryl, heterocyclyl, cycloalkenyl, or cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo.

### Embodiments of R⁵, R⁶, R⁸, and R⁹ in Formula II

In one embodiment of Formula II, at least one of R⁵, R⁶, R⁸, and R⁹ is hydrogen, wherein R¹, R⁴ and R⁷ are as described in Formula II. In another embodiment of Formula II, at least two of R⁵, R⁶, R⁸, and R⁹ are hydrogen, wherein R¹, R⁴ and R⁷ are as described in Formula II. In another embodiment of Formula II, at least three of R⁵, R⁶, R⁸, and R⁹ are hydrogen, wherein R¹, R⁴ and R⁷ are as described in Formula II. In another embodiment of Formula II, at least one of R⁵, R⁶, R⁸, and R⁹ is hydrogen, and the other R⁵, R⁶, R⁸, and R⁹ are independently selected from the group consisting of hydrogen, alkyl, halogen, haloalkyl, —OR¹⁹, and —NR²⁰R²¹; wherein R¹⁹, R²⁰ and R²¹ are selected from the group consisting of hydrogen and alkyl, and wherein R¹, R⁴ and R⁷ are as described in Formula II. In another embodiment of Formula II, at least two of R⁵, R⁶, R⁸, and R⁹ are hydrogen, and the other R⁵, R⁶, R⁸, and R⁹ are independently selected from the group consisting of hydrogen, alkyl, halogen, haloalkyl, —OR¹⁹, and —NR²⁰R²¹; wherein R¹⁹, R²⁰ and R²¹ are selected from the group consisting of hydrogen and alkyl, and wherein R¹, R⁴ and R⁷ are as described in Formula II. In another embodiment of Formula II, at least three of R⁵, R⁶, R⁸, and R⁹ are hydrogen, and the other R⁵, R⁶, R⁸, and R⁹ are independently selected from the group consisting of hydrogen, alkyl, halogen, haloalkyl, —OR¹⁹, and —NR²⁰R²¹; wherein R¹⁹, R²⁰ and R²¹ are selected from the group consisting of hydrogen and alkyl, and wherein R¹, R⁴ and R⁷ are as described in Formula II. In another embodiment of Formula II, at least three of R⁵, R⁶, R⁸, and R⁹ are hydrogen, and the other R⁵, R⁶, R⁸, and R⁹ are independently selected from the group consisting of hydrogen, methyl, ethyl, propyl, fluoro, chloro, bromo, trifluoromethyl, trifluoroethyl, hydroxyl, methoxy, ethoxy, amino, methylamino, and dimethylamino, and wherein R¹, R⁴ and R⁷ are as described in Formula II. In another embodiment of Formula II, at least three of R⁵, R⁶, R⁸, and R⁹ are hydrogen, and the other R⁵, R⁶, R⁸, and R⁹ are independently selected from the group consisting of hydrogen, methyl, fluoro, bromo, hydroxyl, methoxy, and amino, and wherein R¹, R⁴ and R⁷ are as described in Formula II. In another embodiment of Formula II, at least three of R⁵, R⁶, R⁸, and R⁹ are hydrogen, and the other R⁵, R⁶, R⁸, and R⁹ are independently selected from the group consisting of hydrogen and halogen, and wherein R¹, R⁴ and R⁷ are as described in Formula II. In another embodiment of Formula II, at least three of R⁵, R⁶, R⁸, and R⁹ are hydrogen, and the other R⁵, R⁶, R⁸, and R⁹ are independently selected from the group consisting of hydrogen and fluoro, and wherein R¹, R⁴ and R⁷ are as described in Formula II. In one embodiment of Formula II, R⁵, R⁶, R⁸, and R⁹ are hydrogen, as described in Formula III:

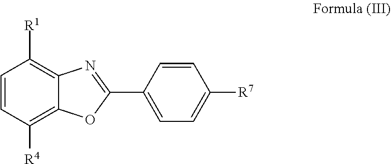

wherein

one of R¹ or R⁴ is —C(O)NH₂ and the other is hydrogen;

when R¹ is —C(O)NH₂; R⁷ is aryl, heterocyclyl, cycloalkyl, cycloalkenyl, alkylamino, alkenyl, alkynyl, halogen, cyano, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —NO₂, —OCF₃, and —OCF₂CF₃;

R¹⁰, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo;

R¹¹ and R¹², at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;

when R⁴ is —C(O)NH₂; R⁷ is aryl, heterocyclyl, cycloalkyl, cycloalkenyl, alkylamino, alkenyl, alkynyl, halogen, cyano, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃;

R¹³, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and

R¹⁴ and R¹⁵, at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo.

### Embodiments of R⁷ in Formula II

In one embodiment of Formula II, R⁷ is selected from the group consisting of aryl and heterocyclyl, wherein R⁷ is optionally substituted as described in Formula II, and R⁵, R⁶, R⁸, and R⁹ are as described in Formula II. In another embodiment of Formula II, R⁷ is selected from the group consisting of aryl and a 3 to 10 membered ring heterocyclyl, wherein R⁷ is optionally substituted as described in Formula II, and R⁵, R⁶, R⁸, and R⁹ are as described in Formula II. In another embodiment of Formula II, R⁷ is selected from the group consisting of aryl and a 4 to 8 membered ring heterocyclyl, wherein R⁷ is optionally substituted as described in Formula II, and R⁵, R⁶, R⁸, and R⁹ are as described in Formula II. In another embodiment of Formula II, R⁷ is selected from the group consisting of aryl and a 5 to 6 membered ring heterocyclyl, wherein R⁷ is optionally substituted as described in Formula II, and R⁵, R⁶, R⁸, and R⁹ are as described in Formula II. In another embodiment, R⁷ is selected from the group consisting of phenyl, pyrrolyl, pyrrolidinyl, imidazolyl, imidazolidinyl, pyrazolyl, pyrazolidinyl, triazolyl, pyridinyl, piperidinyl, morpholinyl, pyridiazinyl, pyrazinyl, and piperidinyl, wherein R⁷ is optionally substituted as described in Formula II, and R⁵, R⁶, R⁸, and R⁹ are as described in Formula II. In another embodiment, R⁷ is selected from the group consisting of halogen, amino, phenyl, pyrazolyl, pyridinyl, piperidinyl, and benzodioxolyl, wherein R⁷ is optionally substituted as described in Formula II, and R⁵, R⁶, R⁸, and R⁹ are as described in Formula II. In another embodiment of Formula II, R⁷ is selected from the group consisting of phenyl,

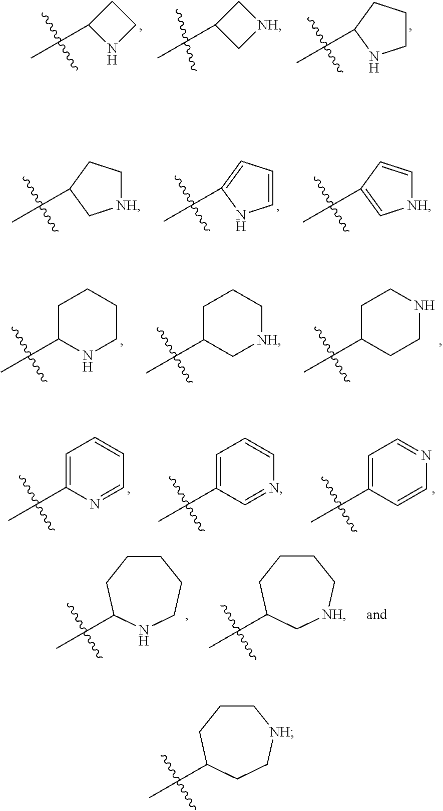

wherein R⁷ is optionally substituted as described in Formula II, and R⁵, R⁶, R⁸, and R⁹ are as described in Formula II. In another embodiment of Formula II, R⁷ is selected from the group consisting of

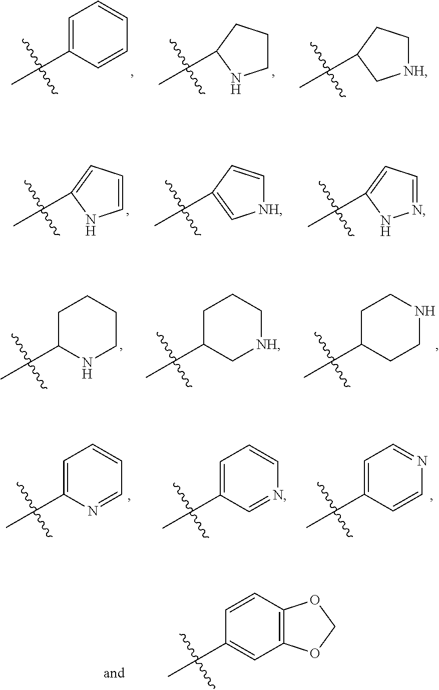

wherein R⁷ is optionally substituted as described in Formula II, and R⁵, R⁶, R⁸, and R⁹ are as described in Formula II. In another embodiment of Formula II, R⁷ is selected from the group consisting of

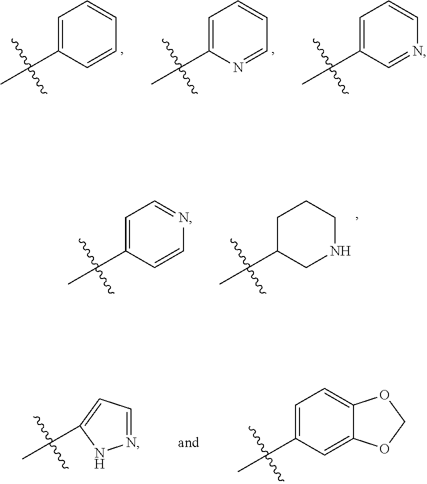

wherein R⁷ is optionally substituted as described in Formula II, and R⁵, R⁶, R⁸, and R⁹ are as described in Formula II. In another embodiment of Formula II, R⁷ is selected from the group consisting of

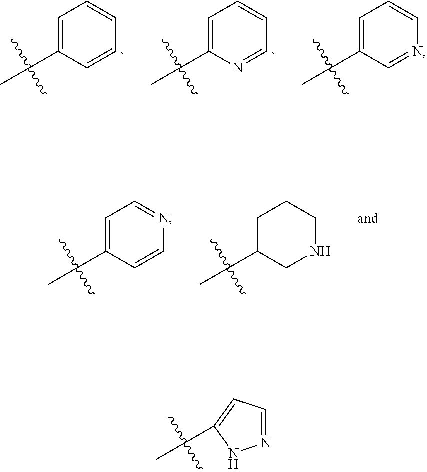

wherein R⁷ is optionally substituted as described in Formula II, and R⁵, R⁶, R⁸, and R⁹ are as described in Formula II. In another embodiment of Formula II, R⁷ is phenyl, wherein R⁷ is optionally substituted as described in Formula II, and R⁵, R⁶, R⁸, and R⁹ are as described in Formula II. In another embodiment of Formula II, R⁷ is pyridinyl, wherein R⁷ is optionally substituted as described in Formula II, and R⁵, R⁶, R⁸, and R⁹ are as described in Formula II.

In another embodiment of Formula II, R⁷ is selected from the group consisting of aryl and heterocyclyl, wherein the aryl and heterocyclyl are optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)R¹⁰, C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —(O)R¹⁰, —SO₂R¹⁰, —OC(O)OR¹⁰, —SO₂NR¹¹R¹², —CF₃, —CF₂CF₃, —N₃, —NO₂, —OCF₃, and —OCF₂CF₃, and R⁵, R⁶, R⁸, and R⁹ are as described in Formula II. In another embodiment of Formula II, R⁷ is selected from the group consisting of aryl and heterocyclyl, wherein the aryl and heterocyclyl are unsubstituted, and R⁵, R⁶, R⁸, R⁹ R¹⁰, R¹¹, and R¹² are as described in Formula II. In another embodiment of Formula II, R⁷ is selected from the group consisting of

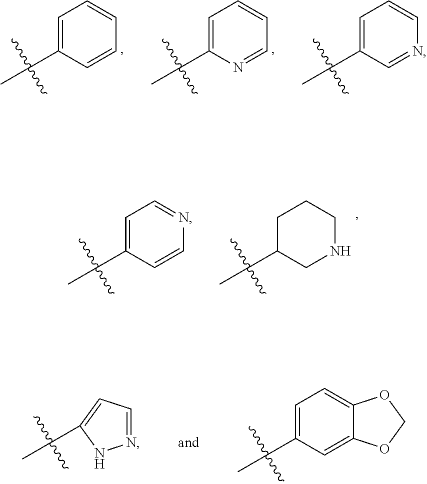

wherein R⁷ is optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², CF₃, —CF₂CF₃, —N₃, —NO₂, —OCF₃, and —OCF₂CF₃, R⁵, R⁶, R⁸, and R⁹ are each hydrogen, and R¹⁰, R¹¹, and R¹² are as described in Formula II. In another embodiment of Formula II, R⁷ is selected from the group consisting of phenyl and pyridinyl, wherein R⁷ is optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)OR¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —NO₂, —OCF₃, and —OCF₂CF₃, and R⁵, R⁶, R⁸, and R⁹ are each hydrogen. In another embodiment of Formula II, R⁷ is selected from the group consisting of phenyl and pyridinyl, wherein R⁷ is optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —NO₂, —OCF₃, and —OCF₂CF₃, and R⁵, R⁶, R⁸, and R⁹ are each hydrogen, and R¹⁰, R¹¹, and R¹² are selected from the group consisting of hydrogen and alkyl. In another embodiment of Formula II, R⁷ is selected from the group consisting of phenyl and pyridinyl, wherein R⁷ is optionally substituted with one or more substituents selected from the group consisting of alkyl, alkenyl, halogen, oxo, —OR¹⁰, —C(O)R¹⁰, —C(O)OR¹⁰, —OC(O)R¹⁰, —NR¹¹R¹², —NR¹¹C(O)R¹², C(O)NR¹¹R¹², —SR¹⁰, —S(O)R¹⁰, —SO₂R¹⁰, —CF₃, and —NO₂, and R⁵, R⁶, R⁸, and R⁹ are each hydrogen, and R¹⁰, R¹¹ and R¹² are selected from the group consisting of hydrogen and alkyl. In another embodiment of Formula II, R⁷ is selected from the group consisting of phenyl and pyridinyl, wherein R⁷ is optionally substituted with one or more substituents selected from the group consisting of alkyl, halogen, oxo, —OR¹⁰, —C(O)R¹⁰, —C(O)OR¹⁰, —OC(O)R¹⁰, NR¹¹R¹², —C(O)NR¹¹R¹², —SR¹⁰, —S(O)R¹⁰, —SO₂R¹⁰, and —CF₃ and R⁵, R⁶, R⁸, and R⁹ are each hydrogen, and R¹⁰, R¹¹ and R¹² are each hydrogen. In another embodiment of Formula II, R⁷ is selected from the group consisting of phenyl and pyridinyl, wherein R⁷ is optionally substituted with one or more substituents selected from the group consisting of alkyl, halogen, oxo, —OR¹⁰, —NR¹¹R¹², and —CF₃ and R⁵, R⁶, R⁸, and R⁹ are each hydrogen, and R¹⁰, R¹¹, and R¹² are each hydrogen. In another embodiment of Formula II, R⁷ is selected from the group consisting of

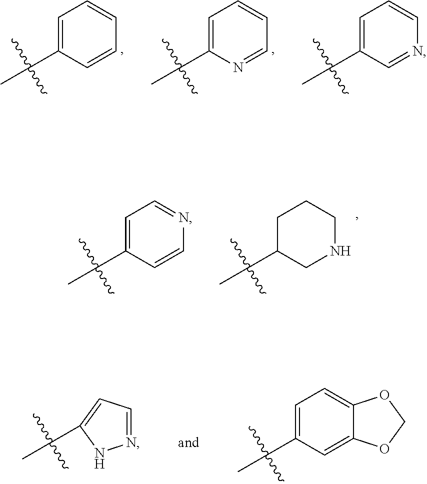

wherein R⁷ is optionally substituted with one —NR¹¹R¹²; R⁵, R⁶, R⁸, and R⁹ are each hydrogen, and R¹¹, and R¹² are each hydrogen.

### Embodiments of Formula V

In one embodiment of Formula I, R¹, R² and R³ are hydrogen, and R⁴ is —C(O)NH₂; as described in Formula IV:

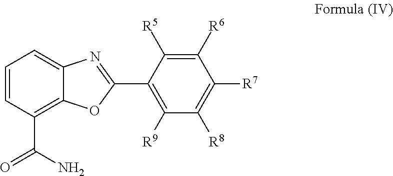

wherein

R⁵, R⁶, R⁸, and R⁹ are independently selected from the group consisting of hydrogen, alkyl, alkenyl, alkynyl, halogen, cyano, haloalkoxy, haloalkyl, hydroxyalkyl, —OR¹⁹, —C(O)OR¹⁹, —NR²⁰R²¹, and —C(O)NR²⁰R²¹;

R⁷ is aryl, heterocyclyl, cycloalkyl, cycloalkenyl, alkylamino, alkenyl, alkynyl, halogen, cyano, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃;

R¹³, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and

R¹⁴ and R¹⁵, at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo.

In one embodiment of Formula IV, R⁵, R⁶, R⁸, and R⁹ are hydrogen as described in Formula V:

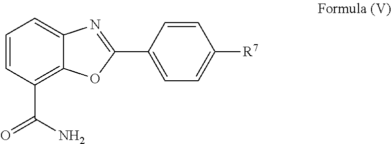

wherein

R⁷ is aryl, heterocyclyl, cycloalkyl, cycloalkenyl, alkylamino, alkenyl, alkynyl, halogen, cyano, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃;

R¹³, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and

R¹⁴ and R¹⁵, at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo.

### Embodiments of R⁷ in Formula V

In one embodiment of Formula V, R⁷ is selected from the group consisting of aryl and heterocyclyl, wherein R⁷ is optionally substituted as described in Formula V. In another embodiment of Formula V, R⁷ is selected from the group consisting of aryl and a 3 to 10 membered ring heterocyclyl, wherein R⁷ is optionally substituted as described in Formula V. In another embodiment of Formula V, R⁷ is selected from the group consisting of aryl and a 4 to 8 membered ring heterocyclyl, wherein R⁷ is optionally substituted as described in Formula V. In another embodiment of Formula VI, R⁷ is selected from the group consisting of aryl and a 5 to 6 membered ring heterocyclyl, wherein R⁷ is optionally substituted as described in Formula V. In another embodiment, R⁷ is selected from the group consisting of phenyl, pyrrolyl, pyrrolidinyl, imidazolyl, imidazolidinyl, pyrazolyl, pyrazolidinyl, triazolyl, pyridinyl, piperidinyl, morpholinyl, pyridiazinyl, pyrasinyl, and piperidinyl, wherein R⁷ is optionally substituted as described in Formula V. In another embodiment, R⁷ is selected from the group consisting of halogen, amino, phenyl, pyrazolyl, pyridinyl, piperidinyl, and benzodioxolyl, wherein R⁷ is optionally substituted as described in Formula V. In another embodiment of Formula V, R⁷ is selected from the group consisting of phenyl,

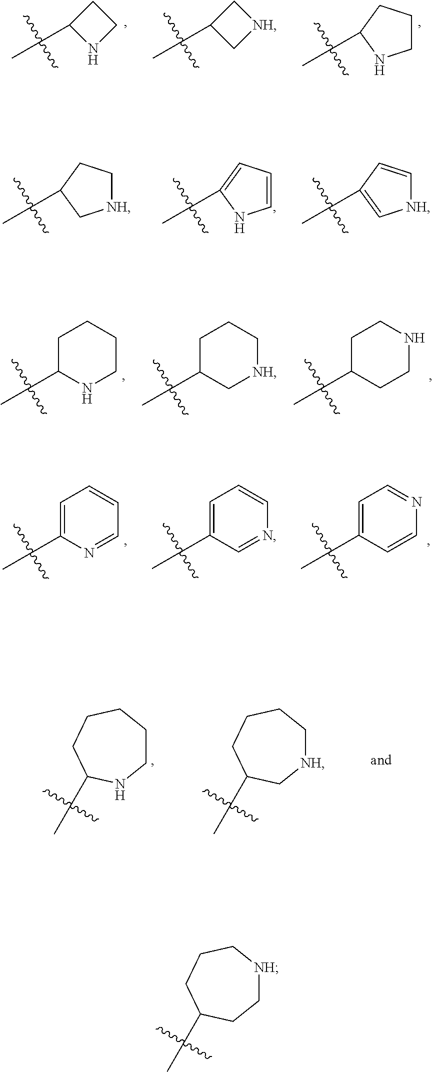

wherein R⁷ is optionally substituted as described in Formula V. In another embodiment of Formula V, R⁷ is selected from the group consisting of

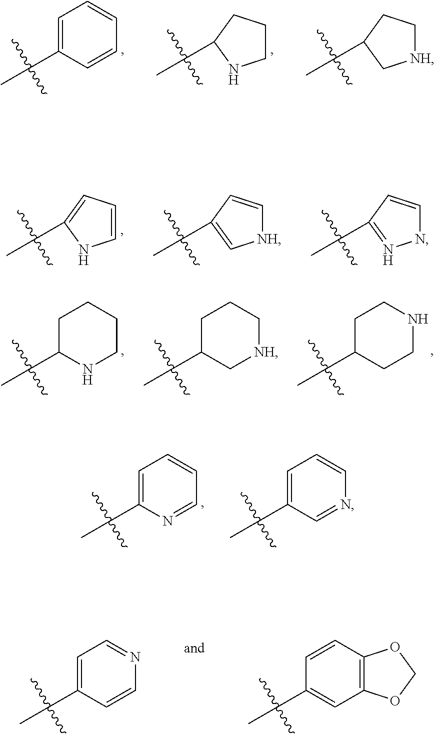

wherein R⁷ is optionally substituted as described in Formula V. In another embodiment of Formula V, R⁷ is selected from the group consisting of

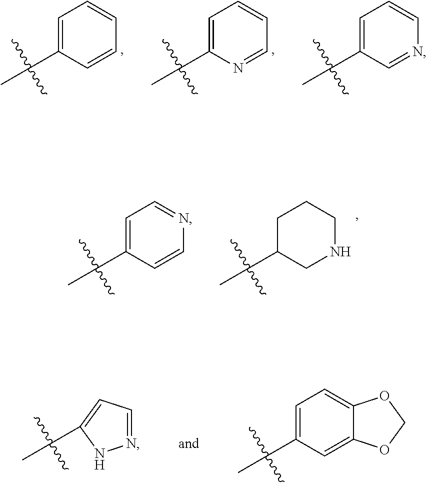

wherein R⁷ is optionally substituted as described in Formula V. In another embodiment of Formula V, R⁷ is selected from the group consisting of

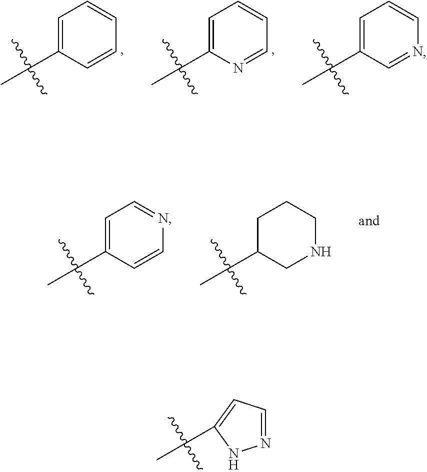

wherein R⁷ is optionally substituted as described in Formula V. In another embodiment of Formula V, R⁷ is phenyl, wherein R⁷ is optionally substituted as described in Formula V. In another embodiment of Formula V, R⁷ is pyridinyl, wherein R⁷ is optionally substituted as described in Formula V.

In another embodiment of Formula V, R⁷ is selected from the group consisting of aryl and heterocyclyl, wherein the aryl and heterocyclyl are substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³—C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃, and R¹³, R¹⁴, and R¹⁵ are as described in Formula V. In another embodiment of Formula V, R⁷ is selected from the group consisting of aryl and heterocyclyl, wherein the aryl and heterocyclyl are unsubstituted. In another embodiment of Formula V, R⁷ is selected from the group consisting of,

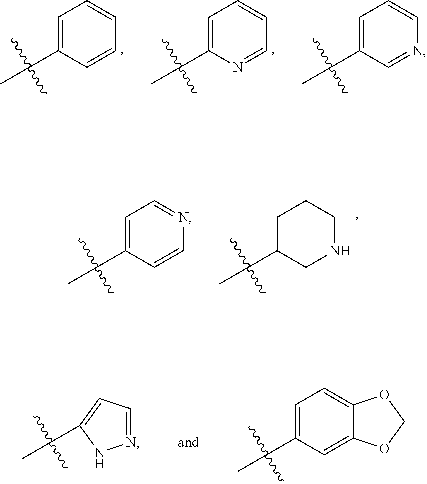

wherein R⁷ is optionally substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃, and R¹³, R¹⁴, and R¹⁵ are as described in Formula V. In another embodiment of Formula V, R⁷ is selected from the group consisting of phenyl and pyridinyl, wherein R⁷ is optionally substituted with one or more substituents selected from the group consisting of aryl, heterocyclyl, cycloalkyl, cycloalkenyl, alkyl, alkenyl alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃, and, and R¹³, R¹⁴, and R¹⁵ are as described in Formula V. In another embodiment of Formula V, R⁷ is selected from the group consisting of phenyl and pyridinyl, wherein R⁷ is optionally substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃, and, and R¹³, R¹⁴, and R¹⁵ are selected from the group consisting of hydrogen and alkyl. In another embodiment of Formula V, R⁷ is selected from the group consisting of phenyl and pyridinyl, wherein R⁷ is optionally substituted with one or more substituents selected from the group consisting of alkyl, alkenyl, halogen, oxo, —OR¹³, —C(O)R¹³, —C(O)OR¹³, —OC(O)R¹³, —NR¹⁴R¹⁵, —NR¹⁴C(O)R¹⁵, —C(O)NR¹⁴R¹⁵, —SR¹³, —S(O)R¹³, —SO₂R¹³, —CF₃, and —NO₂, and, and R¹³, R¹⁴, and R¹⁵ are selected from the group consisting of hydrogen and alkyl. In another embodiment of Formula V, R⁷ is selected from the group consisting of phenyl and pyridinyl, wherein R⁷ is optionally substituted with one or more substituents selected from the group consisting of alkyl, halogen, oxo, —OR¹³, —C(O)R¹³, —C(O)OR¹³, —OC(O)R¹³, —NR¹⁴R¹⁵, —C(O)NR¹⁴R¹⁵, —SR¹³, —S(O)R¹³, —SO₂R¹³, and —CF₃ and, and R¹³, R¹⁴, and R¹⁵ are each hydrogen. In another embodiment of Formula V, R⁷ is selected from the group consisting of phenyl and pyridinyl, wherein R⁷ is optionally substituted with one or more substituents selected from the group consisting of alkyl, halogen, oxo, —OR¹³, —NR¹⁴R¹⁵, and —CF₃, and R¹³, R¹⁴, and R¹⁵ are each hydrogen. In another embodiment of Formula V, R⁷ is selected from the group consisting of

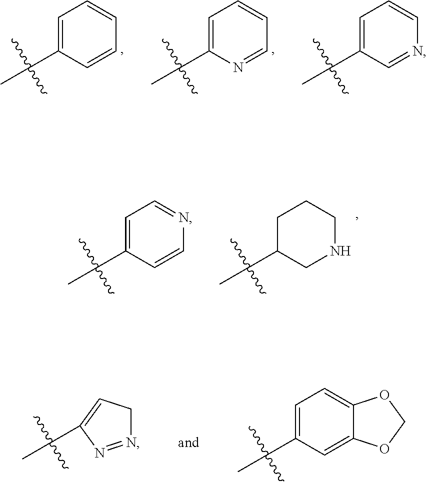

wherein R⁷ is optionally substituted with one —NR¹⁴R¹⁵; and R¹⁴ and R¹⁵ are each hydrogen.

### C. Isomers

This invention also is directed, in part, to all isomers of the compounds of Formula I (and their salts) (i.e., structural and stereoisomers). Structural isomers include chain and position isomers. Stereoisomers include E/Z isomers (i.e., isomers with regard to one or more double bonds), enantiomers (i.e., stereo-isomers that have opposite configurations at all stereogenic centers), and diastereoisomers (i.e., stereo-isomers that have the same configuration at one or more stereogenic centers, but differ at other stereogenic centers). Compounds of this invention may contain asymmetrically substituted carbon atoms in the R or S configuration, wherein the terms “R” and “S” are as defined in Pure Appl. Chem. (1976) 45, 13-10. Accordingly, this invention is meant to embrace racemic mixtures, relative and absolute diastereoisomers and the compounds thereof.

### D. Salts

This invention also is directed, in part, to all salts of the compounds of Formula I. A salt of a compound may be advantageous due to one or more of the salt's properties, such as, for example, enhanced pharmaceutical stability in differing temperatures and humidities, or a desirable solubility in water or other solvents. Where a salt is intended to be administered to a patient (as opposed to, for example, being in use in an in vitro context), the salt preferably is pharmaceutically acceptable and/or physiologically compatible. The term “pharmaceutically acceptable” is used adjectivally in this patent application to mean that the modified noun is appropriate for use as a pharmaceutical product or as a part of a pharmaceutical product. Pharmaceutically acceptable salts include salts commonly used to form alkali metal salts and to form addition salts of free acids or free bases. In general, these salts typically may be prepared by conventional means by reacting, for example, the appropriate acid or base with a compound of the invention.

Pharmaceutically acceptable acid addition salts of the compounds of Formula I can be prepared from an inorganic or organic acid. Examples of often suitable inorganic acids include hydrochloric, hydrobromic, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Specific examples of often suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), ethanesulfonate, benzenesulfonate, pantothenate, 2-hydroxyethanesulfonate, sulfanilate, cyclohexylaminosulfonate, algenic acid, beta-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, heptanoate, hexanoate, nicotinate, oxalate, palmoate, pectinate, 2-naphthalesulfonate, 3-phenylpropionate, picrate, pivalate, thiocyanate, tosylate, and undecanoate.

Pharmaceutically acceptable base addition salts of the compounds of Formula I include, for example, metallic salts and organic salts. Preferred metallic salts include alkali metal (group Ia) salts, alkaline earth metal (group IIa) salts, and other physiologically acceptable metal salts. Such salts may be made from aluminum, calcium, lithium, magnesium, potassium, sodium, and zinc. Preferred organic salts can be made from amines, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

### E. Purity

Compounds of formula I (and salts thereof) with any level of purity (including pure and substantially pure) are within the scope of Applicants' invention. The term “substantially pure” in reference to a compound/salt/isomer, means that the preparation/composition containing the compound/salt/isomer contains more than about 85% by weight of the compound/salt/isomer, preferably more than about 90% by weight of the compound/salt/isomer, preferably more than about 95% by weight of the compound/salt/isomer, preferably more than about 97% by weight of the compound/salt/isomer, and preferably more than about 99% by weight of the compound/salt/isomer.

### F. Methods for Preparation of the Compounds and Salts

The starting materials used herein are commercially available or may be prepared by routine methods well known to those of ordinary skill in the art. The exemplified compounds were named using ACD/ChemSketch Version 5.06 (5 Jun. 2001, Advanced Chemistry Development Inc., Toronto, Ontario).

### Example 1

### 2-(4-bromophenyl)-1,3-benzoxazole-4-carboxamide

### Example 1A

### 2-(4-bromobenzamido)-3-hydroxybenzoic acid

To a suspension of 2-amino-3-hydroxybenzoic acid (2.5 g) in toluene (83 mL) and pyridine (3.3 mL) was added 4-bromobenzoyl chloride (7.17 g) and the mixture heated at 70° C. for 1 hour. The mixture was cooled, diluted with ethyl acetate (40 mL), and washed with 1% hydrochloric acid and brine. The aqueous layer was filtered to afford the title compound. The organic layer was washed with brine, dried over magnesium sulfate and concentrated to give additional title compound.

### Example 1B

### 2-(4-bromophenyl)benzo[d]oxazole-4-carboxylic acid

To a solution of EXAMPLE 1A (3.11 g) in p-xylene (120 mL) was added p-toluenesulfonic acid (4.82 g) and the mixture was heated at 140° C. for 3 hours. The mixture was cooled, diluted with ethyl acetate and washed with 5% citric acid. The organic layer was dried over sodium sulfate, filtered, and partially concentrated. The solid that precipitated out of the organic layer was filtered and dried to afford the title compound.

### Example IC

### 2-(4-bromophenyl)benzo[d]oxazole-4-carboxamide

To a solution of EXAMPLE 1B (0.80 g) in N,N-dimethylformamide (32 mL) was added N-hydroxybenzotriazole (0.578 g) and 1-ethyl-3-(3-dimethylaminopropyl)carbodiimide (0.723 g) and the mixture stirred at ambient temperature for 30 minutes. Concentrated ammonium hydroxide (7.05 mL) was added and mixture stirred at ambient temperature for 6 hours. Water was added and the precipitate was filtered and dried to afford the title compound. ¹H NMR (DMSO-d₆) 8.44 (s, 1H), 8.24-8.28 (m, 2H), 8.03 (s, 1H), 7.98-8.04 (m, 2H), 7.85-7.88 (m, 2H), 7.57 (t, J=7.9 Hz, 1H).

### Example 2

### 2-(1,1′-biphenyl-4-yl)-1,3-benzoxazole-4-carboxamide

A mixture of EXAMPLE 1C (0.03 g), phenylboronic acid (0.013 g), dichlorobis(triphenylphosphine)palladium(II) (6.64 mg) and 1M sodium carbonate (0.13 mL) in 7:2:3 dimethoxyethane/ethanol/water (1 mL) was heated in a microwave at 160° C. for 10 minutes. The crude material was purified by preparative HPLC using a gradient of 10%-100% acetonitrile/water/0.1% trifluoroacetic acid to afford the title compound as the trifluoroacetate salt. ¹H NMR (DMSO-d₆) 12.50 (s, 1H), 9.64 (s, 1H), 8.53 (d, J=8.5 Hz, 2H), 7.86 (d, J=8.5 Hz, 2H), 7.80 (d, J=7.3 Hz, 2H), 7.59 (dd, J=7.9, 1.5 Hz, 1H), 7.52 (t, J=7.6 Hz, 2H), 7.44 (d, J=7.3 Hz, 1H), 7.35 (t, J=7.8 Hz, 1H), 7.24 (dd, J=7.9, 1.2 Hz, 1H), 6.52 (s, 1H).

### Example 3

### 2-(4-pyridin-3-ylphenyl)-1,3-benzoxazole-4-carboxamide

The title compound was prepared as a trifluoroacetate salt as described in EXAMPLE 2 substituting pyridin-3-ylboronic acid for phenylboronic acid. ¹H NMR (DMSO-d₆) 12.54 (s, 1H), 9.67 (s, 1H), 9.10 (d, J=1.8 Hz, 1H), 8.69 (d, J=4.9 Hz, 1H), 8.59 (d, J=8.5 Hz, 2H), 8.37 (d, J=8.2 Hz, 2H), 7.97 (d, J=8.2 Hz, 1H), 7.66 (dd, J=7.8, 1.4 Hz, 1H), 7.36 (t, J=7.9 Hz, 1H), 7.25 (dd, J=7.8, 1.4 Hz, 1H).

### Example 4

### 2-(4-bromophenyl)-1,3-benzoxazole-7-carboxamide

### Example 4A

### 3-(4-bromobenzamido)-2-hydroxybenzoic acid

A suspension of 3-amino-2-hydroxybenzoic acid (1.5 g) in toluene (50 mL) and pyridine (2 mL) was treated with 4-bromobenzoyl chloride (2.58 g) and the mixture was heated at 70° C. for 1 hour. The mixture was cooled and filtered. The solid was washed with ethyl acetate, stirred in water for 15 minutes, filtered and dried to afford the title compound.

### Example 4B

### 2-(biphenyl-4-yl)benzo[d]oxazole-4-carboxamide

The title compound was prepared as described in EXAMPLE 1B, substituting EXAMPLE 4A for EXAMPLE 1A.

### Example 4C

### 2-(4-bromophenyl)benzo[d]oxazole-7-carboxamide

The title compound was prepared as described in EXAMPLE 1C, substituting EXAMPLE 4B for EXAMPLE 1B. ¹H NMR (DMSO-d₆) 8.23-8.26 (m, 2H), 7.98 (d, J=7.9 Hz, 1H), 7.91 (s, 1H), 7.83-7.88 (m, 4H), 7.51 (t, J=7.9 Hz, 1H).

### Example 5

### 2-(1,1′-biphenyl-4-yl)-1,3-benzoxazole-7-carboxamide

A mixture of EXAMPLE 4C (80 mg), phenylboronic acid (34 mg), dichlorobis(triphenylphosphine)palladium(II) (18 mg) and 1M sodium carbonate (0.35 mL) in 7:2:3 dimethoxyethane/ethanol/water (3 mL) was heated in a microwave at 160° C. for 10 minutes. The crude material was purified by preparative HPLC using a gradient of 10%-100% acetonitrile/water/0.1% trifluoroacetic acid to afford the title compound. ¹H NMR (DMSO-d₆) 13.97 (s, 1H), 9.51 (s, 1H), 8.55 (s, 1H), 8.07 (d, J=8.3 Hz, 2H), 7.99 (d, J=7.9 Hz, 1H), 7.84 (d, J=8.3 Hz, 2H), 7.76 (d, J=7.1 Hz, 1H), 7.72 (dd, J=7.9, 1.6 Hz, 1H), 7.49-7.54 (m, 1H), 7.43-7.45 (m, 1H), 6.91 (t, J=7.9 Hz, 1H).

### Example 6

### 2-[4-(1,3-benzodioxol-5-yl)phenyl]-1,3-benzoxazole-7-carboxamide

The title compound was prepared as described in EXAMPLE 5, substituting benzo[d][1,3]dioxol-5-ylboronic acid for phenylboronic acid. ¹H NMR (DMSO-d₆) 13.97 (s, 1H), 9.47 (s, 1H), 8.56 (d, J=1.6 Hz, 1H), 8.02 (d, J=8.3 Hz, 3H), 7.78 (d, J=8.7 Hz, 2H), 7.72 (dd, J=8.1, 1.8 Hz, 1H), 7.36 (d, J=1.6 Hz, 1H), 7.27 (dd, J=8.1, 1.8 Hz, 1H), 7.04 (d, J=7.9 Hz, 1H), 6.91 (t, J=7.9 Hz, 1H), 6.09 (s, 2H).

### Example 7

### 2-[4-(1H-pyrazol-3-yl)phenyl]-1,3-benzoxazole-7-carboxamide

The title compound was prepared as the trifluoroacetate salt as described in EXAMPLE 5 substituting 3-(4,4,5,5-tetramethyl-1,3,2-dioxaborolan-2-yl)-1H-pyrazole for phenylboronic acid. ¹H NMR (DMSO-d₆) 13.97 (s, 1H), 9.40 (s, 1H), 8.55 (s, 1H), 8.19 (s, 2H), 8.06 (s, 1H), 7.96-7.99 (m, 3H), 7.77 (d, J=8.5 Hz, 2H), 7.71 (dd, J=8.2, 1.5 Hz, 1H), 6.90 (t, J=7.9 Hz, 1H).

### Example 8

### 2-(3′-amino-1,1′-biphenyl-4-yl)-1,3-benzoxazole-7-carboxamide

The title compound was prepared as described in EXAMPLE 5 substituting 3-aminobenzeneboronic acid for phenylboronic acid. ¹H NMR (DMSO-d₆) 13.98 (s, 1H), 9.48 (s, 1H), 8.55 (s, 1H), 8.05 (d, J=8.5 Hz, 3H), 7.98-8.01 (m, 1H), 7.74 (d, J=8.5 Hz, 3H), 7.26 t, J=8.0 Hz, 1H), 7.08-7.10 (m, 2H), 6.91 (t, J=8.0 Hz, 1H), 6.80-6.82 (m, 1H).

### Example 9

### 2-(4-pyridin-3-ylphenyl)-1,3-benzoxazole-7-carboxamide

The title compound was prepared as the trifluoroacetate salt as described in EXAMPLE 5, substituting pyridin-3-ylboronic acid for phenylboronic acid. ¹H NMR (DMSO-d₆) 13.98 (s, 1H), 9.59 (s, 1H), 9.10 (d, J=1.8 Hz, 1H), 8.72 (dd, J=5.0, 1.4 Hz, 1H), 8.41 (d, J=7.9 Hz, 1H), 8.13 (d, J=8.5 Hz, 2H), 7.95-7.97 (m, 3H), 7.71-7.74 (m, 2H), 6.92 (t, J=7.9 Hz, 1H).

### Example 10

### 2-(4-pyridin-4-ylphenyl)-1,3-benzoxazole-7-carboxamide

The title compound was prepared as the trifluoroacetate salt as described in EXAMPLE 5, substituting pyridin-4-ylboronic acid for phenylboronic acid. ¹H NMR (DMSO-d₆) 13.98 (s, 1H), 9.65 (s, 1H), 8.80 (d, J=6.4 Hz, 2H), 8.57 (s, 1H), 8.15 (d, J=8.2 Hz, 2H), 8.03-8.06 (m, 3H), 7.94-7.97 (m, 1H), 7.74 (dd, J=7.9, 1.5 Hz, 1H), 6.92 (t, J=7.9 Hz, 1H).

### Example 11

### 2-(4-pyridin-2-ylphenyl)-1,3-benzoxazole-7-carboxamide

To a mixture of EXAMPLE 4C (45 mg), tris(dibenzylideneacetone) dipalladium(0) (13 mg) and tri-o-tolylphosphine (14 mg) was added 2-(tributylstannyl)pyridine (0.061 mL) in N,N-dimethylformamide (2 mL) and triethylamine (0.015 mL) and the mixture stirred at 90° C. for 3 days. Due to significant starting material remaining, additional reagents were added and the mixture stirred an additional 3 hours. Water was added and the mixture was extracted with ethyl acetate. The organic layer was dried over magnesium sulfate and concentrated. The precipitated material was filtered and purified by preparative HPLC using a gradient of 10%-100% acetonitrile/water/0.1% trifluoroacetic acid to afford the title compound as the trifluoroacetate salt. ¹H NMR (DMSO-d₆) 8.75 (d, J=4.0 Hz, 1H), 8.42-8.44 (m, 2H), 8.36-8.37 (m, 2H), 8.13 (d, J=8.2 Hz, 1H), 7.99-8.0 (m, 1H), 7.96 (dd, J=7.9, 1.8 Hz, 2H), 7.87 (s, 1H), 7.83-7.85 (m, 1H), 7.52 (t, J=7.9 Hz, 1H), 7.45 (dd, J=6.6, 4.7 Hz, 1H).

### Example 12

### 2-phenyl-1,3-benzoxazole-4-carboxamide

The title compound was prepared as described in EXAMPLE 1 substituting benzoyl chloride for 4-bromobenzoyl chloride. ¹H NMR (DMSO-d₆) 8.47 (s, 1H), 8.33 (dd, J=8.1, 1.7 Hz, 2H), 7.97-8.03 (m, 3H), 7.64-7.71 (m, 3H), 7.56 (t, J=7.8 Hz, 1H).

### Example 13

### 2-(4-piperidin-3-ylphenyl)-1,3-benzoxazole-7-carboxamide

To a solution of EXAMPLE 9 (40 mg) in ethanol (0.5 mL) was added 5% palladium on carbon and the mixture was heated at 50° C. for 1 hour. The mixture was cooled and filtered over celite. The filtrate was concentrated and purified by preparative HPLC using a gradient of 10%-100% acetonitrile/water/0.1% trifluoroacetic acid to afford the title compound as the trifluoroacetate salt. ¹H NMR (MeOH-d₄) 8.23 (dd, J=8.0, 1.5 Hz, 1H), 7.96 (d, J=8.3, 2H), 7.59 (dd, J=8.3, 1.5 Hz, 1H), 7.48 (d, J=8.3 Hz, 2H), 6.93 (t, J=8.1 Hz, 1H), 3.44-3.48 (m, 2H), 3.11-3.16 (m, 2H), 2.08-2.09 (m, 2H), 1.87-1.93 (m, 2H).

### G. Compositions

This invention also is directed, in part, to compositions comprising one or more compounds and/or salts of the in invention. The compositions can be pharmaceutical compositions.

In some embodiments, the compositions further comprise one or more additional therapeutic agents. Such therapeutic agents can be additional anti-cancer agents.

The preferred composition depends on the method of administration, and typically comprises one or more conventional pharmaceutically acceptable carriers, adjuvants, and/or vehicles (together referred to as “excipients”). Formulation of drugs is generally discussed in, for example, Hoover, J., Remington's Pharmaceutical Sciences (Mack Publishing Co., 1975) and Ansel's Pharmaceutical Dosage Forms and Drug Delivery Systems (Lippincott Williams & Wilkins, 2005).

Solid dosage forms for oral administration include, for example, capsules, tablets, pills, powders, and granules. In such solid dosage forms, the compounds or salts are ordinarily combined with one or more excipients. If administered per os, the compounds or salts can be mixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in, for example, a dispersion of the compound or salt in hydroxypropylmethyl cellulose. In the case of capsules, tablets, and pills, the dosage forms also can comprise buffering agents, such as sodium citrate, or magnesium or calcium carbonate or bicarbonate. Tablets and pills additionally can be prepared with enteric coatings. Liquid dosage forms for oral administration include, for example, pharmaceutically-acceptable emulsions (including both oil-in-water and water-in-oil emulsions), solutions (including both aqueous and non-aqueous solutions), suspensions (including both aqueous and non-aqueous suspensions), syrups, and elixirs containing inert diluents commonly used in the art (e.g., water). Such compositions also can comprise, for example, wetting, emulsifying, suspending, flavoring (e.g., sweetening), and/or perfuming agents.

Parenteral administration includes subcutaneous injections, intravenous injections, intramuscular injections, intrasternal injections, and infusion. Injectable preparations (e.g., sterile injectable aqueous or oleaginous suspensions) can be formulated according to the known art using suitable dispersing, wetting agents, and/or suspending agents. Acceptable vehicles and solvents include, for example, water, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), and/or polyethylene glycols.

Formulations for parenteral administration may, for example, be prepared from sterile powders or granules having one or more of the excipients mentioned for use in the formulations for oral administration. A compound or salt of the invention can be dissolved in water, polyethylene glycol, propylene glycol, ethanol, corn oil, cottonseed oil, peanut oil, sesame oil, benzyl alcohol, sodium chloride, and/or various buffers. The pH may be adjusted, if necessary, with a suitable acid, base, or buffer.

Suppositories for rectal administration can be prepared by, for example, mixing a compound or salt of the invention with a suitable nonirritating excipient that is solid at ordinary temperatures, but liquid at the rectal temperature, and will therefore melt in the rectum to release the drug. Suitable excipients include, for example, cocoa butter; synthetic mono-, di-, or triglycerides, fatty acids, and/or polyethylene glycols.

Topical administration includes the use of transdermal administration, such as transdermal patches or iontophoresis devices.

Other excipients and modes of administration known in the pharmaceutical art also may be used.

The preferred total daily dose of the compound or salt (administered in single or divided doses) is typically from about 0.001 to about 100 mg/kg, more preferably from about 0.001 to about 30 mg/kg, and even more preferably from about 0.01 to about 10 mg/kg (i.e., mg of the compound or salt per kg body weight). Dosage unit compositions can contain such amounts or submultiples thereof to make up the daily dose. In many instances, the administration of the compound or salt will be repeated a plurality of times. Multiple doses per day typically may be used to increase the total daily dose, if desired.

Factors affecting the preferred dosage regimen include the type, age, weight, sex, diet, and condition of the patient; the severity of the pathological condition; the severity of the pathological condition; the route of administration; pharmacological considerations, such as the activity, efficacy, pharmacokinetic, and toxicology profiles of the particular compound or salt used; whether a drug delivery system is utilized; and whether the compound or salt is administered as part of a drug combination. Thus, the dosage regimen actually employed can vary widely, and therefore, can derive from the preferred dosage regimen set forth above.

### H. Kits

This invention also is directed, in part, to a kit comprising one or more compounds and/or salts of the in invention. The kit can optionally contain one or more additional therapeutic agents and/or instructions for, for example, using the kit.

### I. Methods of Use

As PARP inhibitors, the compounds of this invention have numerous therapeutic applications related to ischemia reperfusion injury, inflammatory diseases, degenerative diseases, protection from adverse effects of cytotoxic compounds, and potentiation of cytotoxic cancer therapy. In one embodiment, the treatment is palliative treatment. In another embodiment, the treatment is preventative. In another embodiment, the treatment is restorative. In particular, compounds of this invention potentiate radiation and chemotherapy by increasing cell death of cancer cells, limiting tumor growth, decreasing metastasis, and prolonging the survival of tumor-bearing mammals. Compounds having formula I can treat leukemia, colon cancer, lung cancer, glioblastomas, lymphomas, melanomas, carcinomas of the breast or prostate, and cervical carcinomas. Still another embodiment comprises methods of treating ischemia reperfusion injury associated with myocardial infarction, stroke, neural trauma or organ transplantation in a mammal comprising administering thereto a therapeutically acceptable amount of a compound having formula I. Still another embodiment comprises methods of treating reperfusion of the eye, kidney, gut or skeletal muscle in a mammal comprising administering thereto a therapeutically acceptable amount of a compound having formula I. Still another embodiment comprises methods of treating arthritis, gout, inflammatory bowel disease, CNS inflammation, multiple sclerosis, allergic encephalitis, sepsis, septic shock, hemmorhagic shock, pulmonary fibrosis or uveitis in a mammal comprising administering thereto a therapeutically acceptable amount of a compound having formula I. Still another embodiment comprises a method of treating rheumatoid arthritis or septic shock in a mammal comprising administering thereto a therapeutically acceptable amount of a compound having formula I. Still another embodiment comprises methods of treating diabetes or Parkinsons disease in a mammal comprising administering thereto a therapeutically acceptable amount of a compound having formula I. Still another embodiment comprises methods of treating hypoglycemia in a mammal comprising administering thereto a therapeutically acceptable amount of a compound having formula I. Still another embodiment comprises methods of treating retroviral infection in a mammal comprising administering thereto a therapeutically acceptable amount of a compound having formula I. Still another embodiment comprises methods of treating liver toxicity following acetominophen overdose in a mammal comprising administering thereto a therapeutically acceptable amount of a compound having formula I. Still another embodiment comprises a method of treating cardiac or kidney toxicities from doxorubicin or platinum based antineoplastic agents in a mammal comprising administering thereto a therapeutically acceptable amount of a compound having formula I. Still another embodiment comprises methods of treating skin damage secondary to sulfur mustards in a mammal comprising administering thereto a therapeutically acceptable amount of a compound having formula I.

Compounds having formula I are also expected to be useful when used with alkylating agents, angiogenesis inhibitors, antibodies, antimetabolites, antimitotics, antiproliferatives, aurora kinase inhibitors, Bcr-Abl kinase inhibitors, biologic response modifiers, cyclin-dependent kinase inhibitors, cell cycle inhibitors, cyclooxygenase-2 inhibitors, leukemia viral oncogene homolog (ErbB2) receptor inhibitors, growth factor inhibitors, heat shock protein (HSP)-90 inhibitors, histone deacetylase (HDAC) inhibitors inhibitors, hormonal therapies, immunologicals, intercalating antibiotics, kinase inhibitors, mammalian target of rapomycin inhibitors, mitogen-activated extracellular signal-regulated kinase inhibitors, non-steroidal anti-inflammatory drugs (NSAID's), platinum chemotherapeutics, polo-like kinase inhibitors, proteasome inhibitors, purine analogs, pyrimidine analogs, receptor tyrosine kinase inhibitors, retinoids/deltoids plant alkaloids, topoisomerase inhibitors and the like.

Alkylating agents include altretamine, AMD-473, AP-5280, apaziquone, bendamustine, brostallicin, busulfan, carboquone, carmustine (BCNU), chlorambucil, Cloretazine™ (VNP 40101M), cyclophosphamide, decarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, treosulfan, trofosfamide and the like.

Angiogenesis inhibitors include endothelial-specific receptor tyrosine kinase (Tie-2) inhibitors, epidermal growth factor receptor (EGFR) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs vascular endothelial growth factor receptor tyrosine kinase (VEGFR) inhibitors and the like.

Aurora kinase inhibitors include AZD-1152, MLN-8054, VX-680 and the like.

Bcr-Abl kinase inhibitors include DASATINIB® (BMS-354825), GLEEVEC® (imatinib) and the like.

CDK inhibitors include AZD-5438, BMI-1040, BMS-032, BMS-387, CVT-2584, flavopyridol, GPC-286199, MCS-5A, PD0332991, PHA-690509, seliciclib (CYC-202, R-roscovitine), ZK-304709 and the like.

COX-2 inhibitors include ABT-963, ARCOXIA® (etoricoxib), BEXTRA® (valdecoxib), BMS347070, CELEBREX™ (celecoxib), COX-189 (lumiracoxib), CT-3, DERAMAXX® (deracoxib), JTE-522, 4-methyl-2-(3,4-dimethylphenyl)-1-(4-sulfamoylphenyl-1H-pyrrole), MK-663 (etoricoxib), NS-398, parecoxib, RS-57067, SC-58125, SD-8381, SVT-2016, S-2474, T-614, VIOXX® (rofecoxib) and the like.

EGFR inhibitors include ABX-EGF, anti-EGFr immunoliposomes, EGF-vaccine, EMD-7200, ERBITUX® (cetuximab), HR3, IgA antibodies, IRESSA® (gefitinib), TARCEVA® (erlotinib or OSI-774), TP-38, EGFR fusion protein, TYKERB® (lapatinib) and the like.

ErbB2 receptor inhibitors include CP-724-714, CI-1033 (canertinib), Herceptin® (trastuzumab), TYKERB® (lapatinib), OMNITARG® (2C4, petuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her2IgG3, AS HER2 trifunctional bispecfic antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17-AAG-nab, 17-AAG, CNF-101, CNF-1010, CNF-2024, 17-DMAG, geldanamycin, IPI-504, KOS-953, MYCOGRAB®, NCS-683664, PU24FC1, PU-3, radicicol, SNX-2112, STA-9090 VER49009 and the like.

MEK inhibitors include ARRY-142886, ARRY-438162 PD-325901, PD-98059 and the like.

mTOR inhibitors include AP-23573, CCI-779, everolimus, RAD-001, rapamycin, temsirolimus and the like.

Non-steroidal anti-inflammatory drugs include AMIGESIC® (salsalate), DOLOBID® (diflunisal), MOTRIN® (ibuprofen), ORUDIS® (ketoprofen), RELAFEN® (nabumetone), FELDENE® (piroxicam) ibuprofin cream, ALEVE® and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), DAYPRO® (oxaprozin) and the like.

PDGFR inhibitors include C-451, CP-673, CP-868596 and the like.

Platinum chemotherapeutics include cisplatin, ELOXATIN® (oxaliplatin) eptaplatin, lobaplatin, nedaplatin, PARAPLATIN® (carboplatin), satraplatin and the like.

Polo-like kinase inhibitors include BI-2536 and the like.

Thrombospondin analogs include ABT-510, ABT-567, ABT-898, TSP-1 and the like.

VEGFR inhibitors include AVASTIN® (bevacizumab), ABT-869, AEE-788, ANGIOZYME™, axitinib (AG-13736), AZD-2171, CP-547,632, IM-862, Macugen (pegaptamib), NEXAVAR® (sorafenib, BAY43-9006), pazopanib (GW-786034), (PTK-787, ZK-222584), SUTENT® (sunitinib, SU-11248), VEGF trap, vatalanib, ZACTIMA™ (vandetanib, ZD-6474) and the like.

Antimetabolites include ALIMTA® (premetrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflornithine, EICAR, enocitabine, ethnylcytidine, fludarabine, hydroxyurea, 5-fluorouracil (5-FU) alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosate, pelitrexol, pentostatin, raltitrexed, Ribavirin, triapine, trimetrexate, S-1, tiazofurin, tegafur, TS-1, vidarabine, UFT and the like.

Antibiotics include intercalating antibiotics aclarubicin, actinomycin D, amrubicin, annamycin, adriamycin, BLENOXANE® (bleomycin), daunorubicin, CAELYX® or MYOCET® (doxorubicin), elsamitrucin, epirbucin, glarbuicin, ZAVEDOS® (idarubicin), mitomycin C, nemorubicin, neocarzinostatin, peplomycin, pirarubicin, rebeccamycin, stimalamer, streptozocin, VALSTAR® (valrubicin), zinostatin and the like.

Topoisomerase inhibitors include aclarubicin, 9-aminocamptothecin, amonafide, amsacrine, becatecarin, belotecan, BN-80915, CAMPTOSAR® (irinotecan hydrochloride), camptothecin, CARDIOXANE® (dexrazoxine), diflomotecan, edotecarin, ELLENCE® or PHARMORUBICIN® (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include AVASTIN® (bevacizumab), CD40-specific antibodies, chTNT-1/B, denosumab, ERBITUX® (cetuximab), HUMAX-CD4® (zanolimumab), IGF1R-specific antibodies, lintuzumab, PANOREX® (edrecolomab), RENCAREX® (WX G250), RITUXAN® (rituximab), ticilimumab, trastuzimab and the like.

Hormonal therapies include ARIMIDEX® (anastrozole), AROMASIN® (exemestane), arzoxifene, CASODEX® (bicalutamide), CETROTIDE® (cetrorelix), degarelix, deslorelin, DESOPAN® (trilostane), dexamethasone, DROGENIL®, (flutamide), EVISTA® (raloxifene), fadrozole, FARESTON® (toremifene), FASLODEX® (fulvestrant), FEMARA®, (letrozole), formestane, glucocorticoids, HECTOROL® or RENAGEL® (doxercalciferol), lasofoxifene, leuprolide acetate, MEGACE® (megesterol), MIFEPREX® (mifepristone), NILANDRON™ (nilutamide), NOLVADEX® (tamoxifen citrate), PLENAXIS™ (abarelix), predisone, PROPECIA® (finasteride), rilostane, SUPREFACT® (buserelin), TRELSTAR® (luteinizing hormone releasing hormone (LHRH)), vantas, VETORYL®, (trilostane or modrastane), ZOLADEX® (fosrelin, goserelin) and the like.

Deltoids and retinoids include seocalcitol (EB1089, CB1093), lexacalcitrol (KH1060), fenretinide, PANRETIN® (aliretinoin), ATRAGEN® (liposomal tretinoin), TARGRETIN® (bexarotene), LGD-1550 and the like.

Plant alkaloids include, but are not limited to, vincristine, vinblastine, vindesine, vinorelbine and the like.

Proteasome inhibitors include VELCADE® (bortezomib), MG132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, ACTIMMUNE® (interferon gamma-1b), or interferon gamma-n1, combinations thereof and the like. Other agents include ALFAFERONE®, BAM-002, BEROMUN® (tasonermin), BEXXAR® (tositumomab), CamPath® (alemtuzumab), CTLA4 (cytotoxic lymphocyte antigen 4), decarbazine, denileukin, epratuzumab, GRANOCYTE® (lenograstim), lentinan, leukocyte alpha interferon, imiquimod, MDX-010, melanoma vaccine, mitumomab, molgramostim, MYLOTARG™ (gemtuzumab ozogamicin), NEUPOGEN® (filgrastim), OncoVAC-CL, OvaRex® (oregovomab), pemtumomab (Y-muHMFG1), PROVENGE®, sargaramostim, sizofilan, teceleukin, TheraCys®, ubenimex, VIRULIZIN®, Z-100, WF-10, PROLEUKIN® (aldesleukin), ZADAXIN® (thymalfasin), ZENAPAX® (daclizumab), ZEVALIN® (90Y-Ibritumomab tiuxetan) and the like.

Biological response modifiers are agents that modify defense mechanisms of living organisms or biological responses, such as survival, growth, or differentiation of tissue cells to direct them to have anti-tumor activity and include krestin, lentinan, sizofuran, picibanil PF-3512676 (CpG-8954), ubenimex and the like.

Pyrimidine analogs include cytarabine (ara C), cytosine arabinoside, doxifluridine, FLUDARA® (fludarabine), 5-FU (5-fluorouracil), floxuridine, GEMZAR® (gemcitabine), TOMUDEX® (ratitrexed), TROXATYL™ (triacetyluridine troxacitabine) and the like.

Purine analogs include LANVIS® (thioguanine) and PURI-NETHOL® (mercaptopurine).

Antimitotic agents include batabulin, epothilone D (KOS-862), N-(2-((4-hydroxyphenyl)amino)pyridin-3-yl)-4-methoxybenzenesulfonamide, ixabepilone (BMS 247550), paclitaxel, TAXOTERE® (docetaxel), PNU100940 (109881), patupilone, XRP-9881, vinflunine, ZK-EPO and the like.

Compounds of the present invention are also intended to be used as a radiosensitizer that enhances the efficacy of radiotherapy. Examples of radiotherapy include, but are not limited to, external beam radiotherapy, teletherapy, brachtherapy and sealed and unsealed source radiotherapy.

Additionally, compounds having formula I may be combined with other chemptherapeutic agents such as ABRAXANE™ (ABI-007), ABT-100 (farnesyl transferase inhibitor), ADVEXIN®, ALTOCOR® or MEVACOR® (lovastatin), AMPLIGEN® (poly I: poly C12U, a synthetic RNA), APTOSYN™ (exisulind), AREDIA® (pamidronic acid), arglabin, L-asparaginase, atamestane (1-methyl-3,17-dione-androsta-1,4-diene), AVAGE® (tazarotne), AVE-8062, BEC2 (mitumomab), cachectin or cachexin (tumor necrosis factor), canvaxin (vaccine), CeaVac™ (cancer vaccine), CELEUK® (celmoleukin), CEPLENE® (histamine dihydrochloride), CERVARIX™ (human papillomavirus vaccine), CHOP® (C: CYTOXAN® (cyclophosphamide); H: ADRIAMYCIN® (hydroxydoxorubicin); O: Vincristine (ONCOVIN®); P: prednisone), CyPat™, combrestatin A4P, DAB(389)EGF or TransMID-107R™ (diphtheria toxins), dacarbazine, dactinomycin, 5,6-dimethylxanthenone-4-acetic acid (DMXAA), eniluracil, EVIZON™ (squalamine lactate), DIMERICINE® (T4N5 liposome lotion), discodermolide, DX-8951f (exatecan mesylate), enzastaurin, EPO906, GARDASIL® (quadrivalent human papillomavirus (Types 6, 11, 16, 18) recombinant vaccine), gastrimmune, genasense, GMK (ganglioside conjugate vaccine), GVAX® (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, JUNOVAN™ or MEPACT™ (mifamurtide), lonafarnib, 5,10-methylenetetrahydrofolate, miltefosine (hexadecylphosphocholine), NEOVASTAT® (AE-941), NEUTREXIN® (trimetrexate glucuronate), NIPENT® (pentostatin), ONCONASE® (a ribonuclease enzyme), ONCOPHAGE® (melanoma vaccine treatment), OncoVAX (IL-2 Vaccine), ORATHECIN™ (rubitecan), OSIDEM® (antibody-based cell drug), OvaRex® MAb (murine monoclonal antibody), paditaxel, PANDIMEX™ (aglycone saponins from ginseng comprising 20(S)protopanaxadiol (aPPD) and 20(S)protopanaxatriol (aPPT)), panitumumab, PANVAC®-VF (investigational cancer vaccine), pegaspargase, PEG Interferon A, phenoxodiol, procarbazine, rebimastat, REMOVAB® (catumaxomab), REVLIMID® (lenalidomide), RSR13 (efaproxiral), SOMATULINE® LA (lanreotide), SORIATANE® (acitretin), staurosporine (Streptomyces staurospores), talabostat (PT100), TARGRETIN® (bexarotene), Taxoprexin® (DHA-paclitaxel), TELCYTA™ (TLK286), temilifene, TEMODAR® (temozolomide), tesmilifene, thalidomide, THERATOPE® (STn-KLH), thymitaq (2-amino-3,4-dihydro-6-methyl-4-oxo-5-(4-pyridylthio)quinazoline dihydrochloride), TNFerade™ (adenovector: DNA carrier containing the gene for tumor necrosis factor-α), TRACLEER® or ZAVESCA® (bosentan), tretinoin (Retin-A), tetrandrine, TRISENOX® (arsenic trioxide), VIRULIZIN®, ukrain (derivative of alkaloids from the greater celandine plant), vitaxin (anti-alphavbeta3 antibody), XCYTRIN® (motexafin gadolinium), XINLAY™ (atrasentan), XYOTAX™ (paclitaxel poliglumex), YONDELIS™ (trabectedin), ZD-6126, ZINECARD® (dexrazoxane), zometa (zolendronic acid), zorubicin and the like.

In one embodiment, compounds having Formula I are used in a method of treating cancer in a mammal comprising administering thereto a therapeutically acceptable amount of a compound of Formula I in combination with a chemotherapeutic agent selected from temozolomide, dacarbazine, cyclophosphamide, carmustine, melphalan, lomustine, carboplatin, cisplatin, 5-FU+/−leucovorin, gemcitabine, methotrexate, bleomycin, irinotecan, camptothecin, or topotecan.

It is expected that compounds having Formula I would also inhibit growth of cells derived from a pediatric cancer or neoplasm including embryonal rhabdomyosarcoma, pediatric acute lymphoblastic leukemia, pediatric acute myelogenous leukemia, pediatric alveolar rhabdomyosarcoma, pediatric anaplastic ependymoma, pediatric anaplastic large cell lymphoma, pediatric anaplastic medulloblastoma, pediatric atypical teratoid/rhabdoid tumor of the central nervous system, pediatric biphenotypic acute leukemia, pediatric Burkitts lymphoma, pediatric cancers of Ewing's family of tumors such as primitive neuroectodermal rumors, pediatric diffuse anaplastic Wilm's tumor, pediatric favorable histology Wilm's tumor, pediatric glioblastoma, pediatric medulloblastoma, pediatric neuroblastoma, pediatric neuroblastoma-derived myelocytomatosis, pediatric pre-B-cell cancers (such as leukemia), pediatric psteosarcoma, pediatric rhabdoid kidney tumor, pediatric rhabdomyosarcoma, and pediatric T-cell cancers such as lymphoma and skin cancer and the like (commonly-owned U.S. Application Ser No. 10/988,338), Cancer Res., 2000, 60, 6101-10); and autoimmune disorders include, acquired immunodeficiency disease syndrome, autoimmune lymphoproliferative syndrome, hemolytic anemia, inflammatory diseases, thrombocytopenia and the like (Current Allergy and Asthma Reports 2003, 3:378-384; Br. J. Haematol. 2000 September; 110(3): 584-90; Blood 2000 Feb. 15; 95(4):1283-92; and New England Journal of Medicine 2004 September; 351(14): 1409-1418).

### PARP Enzyme Inhihition Assay

Nicotinamide[2,5′,8-3H]adenine dinucleotide and strepavidin SPA beads were purchased from Amersham Biosiences. Recombinant Human Poly(ADP-Ribose) Polymerase (PARP), purified from E. coli and 6-Biotin-17-NAD⁺, were purchase from Trevigen. NAD⁺, histone, aminobenzamide, 3-amino benzamide and Calf Thymus DNA (dcDNA) were purchased from Sigma. Stem loop oligonucleotide containing MCAT sequence was obtained from Qiagen. The oligos were dissoloved to 1 mM in annealing buffer containing 10 mM Tris HCl pH 7.5, 1 mM EDTA, and 50 mM NaCl, incubated for 5 minutes at 95° C., and annealed at 45° C. for 45 minutes. Histone H1 (95% electrophoretically pure) was purchased from Roche. Biotinylated histone H1 was prepared by treating the protein with Sulfo-NHS-LC-Biotin from Pierce. The biotinylation reaction was conducted by slowly and intermittently adding 3 equivalents of 10 mM Sulfo-NHS-LC-Biotin to 100 μM Histone H1 in phosphate-buffered saline, pH 7.5, at 4° C. with gentle vortexing over 1 minute followed by subsequent 4° C. incubation for 1 hour. Streptavidin coated (FlashPlate Plus) microplates were purchased from Perkin Elmer.

PARP1 assay was conducted in PARP assay buffer containing 50 mM Tris pH 8.0, 1 mM DTT, 4 mM MgCl₂. PARP reactions contained 1.5 M [³H]-NAD⁺ (1.6 uCi/mmol), 200 nM biotinylated histone H1, 200 nM slDNA, and 1 nM PARP enzyme. Auto reactions utilizing SPA bead-based detection were carried out in 100 μL volumes in white 96 well plates. Reactions were initiated by adding 50 μl of 2×NAD⁺ substrate mixture to 50 μL of 2× enzyme mixture containing PARP and DNA. These reactions were terminated by the addition of 150 μL of 1.5 mM benzamide (˜1000-fold over its IC50). 170 μL of the stopped reaction mixtures were transferred to streptavidin Flash Plates, incubated for 1 hour, and counted using a TopCount microplate scintillation counter. The IC₅₀ₛ for exemplified compounds of this invention are provided in Table 1.

### Cellular PARP Assay:

C41 cells are treated with a compound of this invention for 30 minutes in 96 well plate. PARP is then activated by damaging DNA with 1 mM H₂O₂ for 10 minutes. The cells are then washed with ice-cold PBS once and fixed with pre-chilled methanol:acetone (7:3) at −20° C. for 10 minutes. After air-drying, the plates are rehydrated with PBS and blocked 5% non-fat dry milk in PBS-TWEEN20® (Sigma, St. Louis, Mo.) (0.05%) (blocking solution) for 30 minutes at room temperature. The cells are incubated with anti-PAR antibody 10H (1:50) in Blocking solution at 37° C. for 60 minutes followed by washing with PBS-TWEEN20® 5 times, and incubation with goat anti-mouse fluorescein 5(6)-isothiocyanate-coupled antibody (1:50) and 1 μg/ml 4′,6-diamidino-2-phenylindole (DAPI) in blocking solution at 37° C. for 60 minutes. After washing with PBS-TWEEN20® 5 times, the analysis is performed using an FMAX FLUORESCENCE MICROPLATE READER® (Molecular Devices, Sunnyvalle, Calif.), set at the excitation wavelength of 490 nm and emission wavelength of 528 nm fluorescein 5(6)-isothiocyanate (FITC) or the excitation wavelength of 355 nm and emission wavelength of 460 nm (DAPI). The PARP activity (FITC signal) is normalized with cell numbers (DAPI).

The cellular assay measures the formation of poly ADP-ribose by PARP within cells and can demonstrate that compounds penetrate cell membranes and inhibit PARP in intact cells.

TABLE 1
Inhibition of PARP by Compounds Having Formula I

| | PARP-1 |
|---|---|
| Example | (Kᵢ, nM) |
| 1 | 142 |
| 2 | 9180 |
| 3 | 312 |
| 4 | 48 |
| 5 | 196 |
| 6 | 841 |
| 7 | 109 |
| 8 | 3250 |
| 9 | 63 |
| 10 | 1620 |
| 11 | 11 |
| 12 | 506 |
| 13 | 757 |

### Cellular PARP Assay:

The cellular assay measures the formation of poly ADP-ribose by PARP within cells and demonstrates that compounds penetrate cell membranes and inhibit PARP in intact cells. C41 cells are treated with a compound of this invention for 30 minutes in 96 well plate. PARP is then activated by damaging DNA with 1 mM H₂O₂ for 10 minutes. The cells are then washed with ice-cold PBS once and fixed with pre-chilled methanol:acetone (7:3) at −20° C. for 10 minutes. After air-drying, the plates are rehydrated with PBS and blocked 5% non-fat dry milk in PBS-TWEEN20® (Sigma, St. Louis, Mo.) (0.05%) (blocking solution) for 30 minutes at room temperature. The cells are incubated with anti-PAR antibody 10H (1:50) in Blocking solution at 37° C. for 60 minutes followed by washing with PBS-TWEEN20® 5 times, and incubation with goat anti-mouse fluorescein 5(6)-isothiocyanate-coupled antibody (1:50) and 1 μg/ml 4′,6-diamidino-2-phenylindole (DAPI) in blocking solution at 37° C. for 60 minutes. After washing with PBS-TWEEN20® 5 times, the analysis is performed using an FMAX FLUORESCENCE MICROPLATE READER® (Molecular Devices, Sunnyvalle, Calif.), set at the excitation wavelength of 490 nm and emission wavelength of 528 nm fluorescein 5(6)-isothiocyanate (FITC) or the excitation wavelength of 355 nm and emission wavelength of 460 nm (DAPI). The PARP activity (FITC signal) is normalized with cell numbers (DAPI).

1. A compound having a structure of Formula I 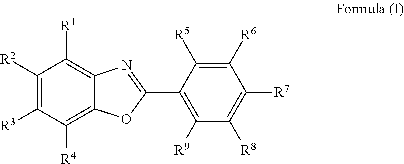
wherein
one of R¹ or R⁴ is —C(O)NH₂ and the other is selected from the group consisting of hydrogen, alkyl, halogen, cyano, haloalkyl, hydroxyalkyl, —OR¹⁶, and —NR¹⁷R¹⁸;
when R¹ is —C(O)NH₂; R⁷ is aryl, heterocyclyl, cycloalkyl, cycloalkenyl, alkylamino, alkenyl, alkynyl, halogen, cyano, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)R¹⁰, —C(O)OR¹⁰, OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —NO₂, —OCF₃, and —OCF₂CF₃;
when R⁴ is —C(O)NH₂; R⁷ is aryl, heterocyclyl, cycloalkyl, cycloalkenyl, alkylamino, alkenyl, alkynyl, halogen, cyano, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃;
R², R³ are independently selected from the group consisting of hydrogen, alkyl, alkenyl, alkynyl, halogen, cyano, haloalkoxy, haloalkyl, hydroxyalkyl, —OR¹⁶, —C(O)OR¹⁶, —NR¹⁷R¹⁸, and —C(O)NR¹⁷R¹⁸;
R⁵, R⁶, R⁸, and R⁹ are independently selected from the group consisting of hydrogen, alkyl, alkenyl, alkynyl, halogen, cyano, haloalkoxy, haloalkyl, hydroxyalkyl, —OR¹⁹, —C(O)OR¹⁹, —NR²⁰R²¹, and —C(O)NR²⁰R²¹;
R¹⁰, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo;
R¹¹ and R¹², at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;
R¹³, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo;
R¹⁴ and R¹⁵, at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;
R¹⁶, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and
R¹⁷ and R¹⁸, at each occurrence, are independently selected from the group consisting of hydrogen, alkyl, alkenyl, alkynyl, aryl, heterocyclyl, cycloalkenyl, or cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo
R¹⁹, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and
R²⁰ and R²¹, at each occurrence, are independently selected from the group consisting of hydrogen, alkyl, alkenyl, alkynyl, aryl, heterocyclyl, cycloalkenyl, or cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;
or a pharmaceutically acceptable salt thereof.

2. The compound of claim 1, wherein R² and R³ are hydrogen.

3. The compound of claim 2, wherein at least three of R⁵, R⁶, R⁸, and R⁹ are hydrogen, and the other R⁵, R⁶, R⁸, or R⁹ is independently selected from the group consisting of hydrogen, methyl, ethyl, propyl, fluoro, chloro, bromo, trifluoromethyl, trifluoroethyl, hydroxyl, methoxy, ethoxy, amino, methylamino, and dimethylamino.

4. The compound of claim 3, wherein R⁷ is selected from the group consisting of aryl and a 4 to 8 membered ring heterocyclyl.

5. The compound of claim 3, wherein R⁷ is selected from the group consisting of phenyl, pyrrolyl, pyrrolidinyl, imidazolyl, imidazolidinyl, pyrazolyl, pyrazolidinyl, triazolyl, pyridinyl, piperidinyl, morpholinyl, pyridiazinyl, pyrazinyl, and piperidinyl.

6. The compound of claim 4, wherein R⁷ is optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —NO₂, —OCF₃, and —OCF₂CF₃.

7. A compound of claim 1 wherein R⁷ is selected from the group consisting of phenyl and pyridinyl, wherein R⁷ is optionally substituted with one or more substituents selected from the group consisting of aryl, heterocyclyl, cycloalkyl, cycloalkenyl, alkyl, alkenyl, alkynyl, halogen, cyano, oxo, —OR¹⁰, —C(O)R¹⁰, —C(O)OR¹⁰, —OC(O)R¹⁰, —NR¹¹R¹², —NR¹¹C(O)R¹², —NHC(O)NHR¹¹, —C(O)NR¹¹R¹², —SR¹⁰, —S(O)R¹⁰, —SO₂R¹⁰, —OC(O)OR¹⁰, —SO₂NR¹¹R¹², —CF₃, —CF₂CF₃, —N₃, —NO₂, —OCF₃, and —OCF₂CF₃, and R⁵, R⁶, R⁸, and R⁹ are each hydrogen.

8. A compound of claim 1 wherein R⁷ is selected from the group consisting of phenyl and pyridinyl, wherein R⁷ is optionally substituted with one or more substituents selected from the group consisting of alkyl, halogen, oxo, —OR¹⁰, —NR¹¹R¹², and —CF₃ and R⁵, R⁶, R⁸, and R⁹ are each hydrogen, and R¹⁰, R¹¹, and R¹² are each hydrogen.

9. A compound of claim 1 wherein R⁷ is selected from the group consisting of 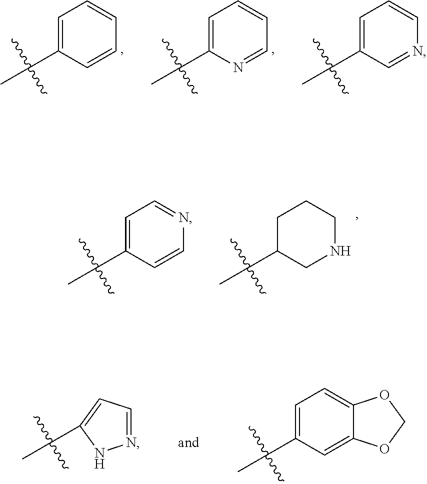
wherein R⁷ is optionally substituted with one —NR¹¹R¹²; R⁵, R⁶, R⁸, and R⁹ are each hydrogen, and R¹¹, and R¹² are each hydrogen.

10. A compound having a structure of Formula IV: 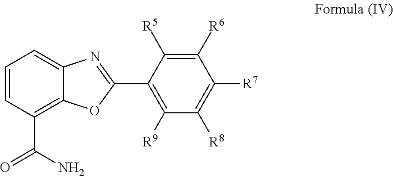
wherein
R⁵, R⁶, R⁸, and R⁹ are independently selected from the group consisting of hydrogen, alkyl, alkenyl, alkynyl, halogen, cyano, haloalkoxy, haloalkyl, hydroxyalkyl, —OR¹⁹, —C(O)OR¹⁹, —NR²⁰R²¹, and —C(O)NR²⁰R²¹;
R⁷ is aryl, heterocyclyl, cycloalkyl, cycloalkenyl, alkylamino, alkenyl, alkynyl, halogen, cyano, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —OCF₃, or —OCF₂CF₃, wherein the aryl, heterocyclyl, cycloalkyl, cycloalkenyl, alkylamino, alkenyl, and alkynyl are optionally substituted with one or more substituents selected from the group consisting of aryl, heterocyclyl, cycloalkyl, cycloalkenyl, alkyl, alkenyl, alkynyl, halogen, cyano, oxo, —OR¹³, —C(O)R¹³, —C(O)OR¹³, —OC(O)R¹³, —NR¹⁴R¹⁵, —NR¹⁴C(O)R¹⁵, —NHC(O)NHR¹⁴, —C(O)NR¹⁴R¹⁵, —SR¹³, —S(O)R¹³, —SO₂R¹³, —OC(O)OR¹³, —SO₂NR¹⁴R¹⁵, —CF₃, —CF₂CF₃, —N₃, —NO₂, —OCF₃, and —OCF₂CF₃;
R¹³, at each occurrence, is independently selected from the group consisting of hydrogen, alkyl, alkenyl, alkynyl, aryl, heterocyclyl, cycloalkyl, wherein the alkyl, alkenyl, alkynyl, aryl, heterocyclyl, and cycloalkyl are optionally substituted with one or more substituents selected from the group consisting aryl, heterocyclyl, cycloalkyl, hydroxyl, halogen, cyano, and oxo; and
R¹⁴ and R¹⁵, at each occurrence, are independently selected from the group consisting of hydrogen, alkyl, alkenyl, alkynyl, aryl, heterocyclyl, cycloalkenyl, and cycloalkyl, wherein the alkyl, alkenyl, alkynyl, heterocyclyl, and cycloalkyl are optionally substituted with one or more substituents selected from the group consisting aryl, heterocyclyl, cycloalkyl, hydroxyl, halogen, cyano, and oxo;
or a pharmaceutically acceptable salt thereof.

11. A compound of claim 10, wherein R⁵, R⁶, R⁸, and R⁹ are hydrogen.

12. A compound of claim 11, wherein R⁷ is selected from the group consisting of phenyl, pyrrolyl, pyrrolidinyl, imidazolyl, imidazolidinyl, pyrazolyl, pyrazolidinyl, triazolyl, pyridinyl, piperidinyl, morpholinyl, pyridiazinyl, pyrasinyl, and piperidinyl.

13. A compound of claim 12, wherein R⁷ is optionally substituted with one or more substituents selected from the group consisting of alkyl, halogen, oxo, —OR³, —NR¹⁴R¹⁵, and —CF₃, and R¹³, R¹⁴, and R¹⁵ are each hydrogen.

14. A compound of claim 12, wherein R⁷ is selected from the group consisting of phenyl and pyridinyl, wherein R⁷ is optionally substituted with one or more substituents selected from the group consisting of alkyl, alkenyl, halogen, oxo, —OR¹³, —C(O)R¹³, —C(O)OR¹³, —OC(O)R¹³, —NR¹⁴R¹⁵, —NR¹⁴C(O)R¹⁵, —C(O)NR¹⁴R¹⁵, —SR¹³, —S(O)R¹³, —SO₂R¹³, —CF₃, and —NO₂, and, and R¹³, R¹⁴, and R¹⁵ are selected from the group consisting of hydrogen and alkyl.

15. A compound of claim 1 selected from the group consisting of
2-(4-bromophenyl)-1,3-benzoxazole-4-carboxamide;
2-(1,1′-biphenyl-4-yl)-1,3-benzoxazole-4-carboxamide;
2-(4-pyridin-3-ylphenyl)-1,3-benzoxazole-4-carboxamide;
2-(4-bromophenyl)-1,3-benzoxazole-7-carboxamide;
2-(1,1′-biphenyl-4-yl)-1,3-benzoxazole-7-carboxamide;
2-[4-(1,3-benzodioxol-5-yl)phenyl]-1,3-benzoxazole-7-carboxamide;
2-[4-(1H-pyrazol-3-yl)phenyl]-1,3-benzoxazole-7-carboxamide;
2-(3′-amino-1,1′-biphenyl-4-yl)-1,3-benzoxazole-7-carboxamide;
2-(4-pyridin-3-ylphenyl)-1,3-benzoxazole-7-carboxamide;
2-(4-pyridin-4-ylphenyl)-1,3-benzoxazole-7-carboxamide;
2-(4-pyridin-2-ylphenyl)-1,3-benzoxazole-7-carboxamide;
2-phenyl-1,3-benzoxazole-4-carboxamide; and
2-(4-piperidin-3-ylphenyl)-1,3-benzoxazole-7-carboxamide;
or a therapeutically acceptable salt thereof.

16. A pharmaceutical composition comprising a compound of claim 1 and pharmaceutically acceptable excipient.

17. A method of treating cancer in a mammal comprising administering thereto a therapeutically acceptable amount of a compound of claim 1.

18. A method for decreasing tumor volume in a mammal comprising administering thereto a therapeutically acceptable amount of a compound of claim 1.

19. A method of treating cancer in a mammal comprising administering thereto a therapeutically acceptable amount of a compound of claim 1 in combination with radiotherapy.

20. A method of treating cancer in a mammal comprising administering thereto a therapeutically acceptable amount of a compound of claim 1 in combination with a chemotherapeutic agent selected from temozolomide, dacarbazine, cyclophosphamide, carmustine, melphalan, lomustine, carboplatin, cisplatin, 5-FU+/−leucovorin, gemcitabine, methotrexate, bleomycin, irinotecan, camptothecin, or topotecan.
